SEQUENCE LISTING (41 sequences)

SEQ ID NO: 1 - protein, 15 aa, Artificial Sequence
  Description of Artificial Sequence Synthetic peptide
KLFLESGAVKYLEGH

SEQ ID NO: 2 - protein, 10 aa, Artificial Sequence
  Description of Artificial Sequence Synthetic peptide
KTVLDHILQK

SEQ ID NO: 3 - protein, 18 aa, Artificial Sequence
  Description of Artificial Sequence Synthetic peptide
HNSTLQVSDVDKLVCRDK

SEQ ID NO: 4 - protein, 10 aa, Artificial Sequence
  Description of Artificial Sequence Synthetic peptide
KAALSSLAKH

SEQ ID NO: 5 - protein, 14 aa, Artificial Sequence
  Description of Artificial Sequence Synthetic peptide
KGFLCDSSFCKKDH

SEQ ID NO: 6 - protein, 10 aa, Artificial Sequence
  Description of Artificial Sequence Synthetic peptide
KKILMNFHQK

SEQ ID NO: 7 - protein, 9 aa, Artificial Sequence
  Description of Artificial Sequence Synthetic peptide
KILMNFHQK

SEQ ID NO: 8 - protein, 13 aa, Artificial Sequence
  Description of Artificial Sequence Synthetic peptide
KITLLTLIKTAEH

SEQ ID NO: 9 - protein, 10 aa, Artificial Sequence
  Description of Artificial Sequence Synthetic peptide
KILEQFHLQK

SEQ ID NO: 10 - protein, 13 aa, Artificial Sequence
  Description of Artificial Sequence Synthetic peptide
KGGQQKNSQKGQH

SEQ ID NO: 11 - protein, 18 aa, Artificial Sequence
  Description of Artificial Sequence Synthetic peptide
HHAYQGDYKLFLESGAVK

SEQ ID NO: 12 - protein, 14 aa, Artificial Sequence
  Description of Artificial Sequence Synthetic peptide
HGFRFEVKKRDGVK

SEQ ID NO: 13 - protein, 10 aa, Artificial Sequence
  Description of Artificial Sequence Synthetic peptide
KVKKHATVLK

SEQ ID NO: 14 - protein, 8 aa, Artificial Sequence
  Description of Artificial Sequence Synthetic peptide
KKHATVLK

SEQ ID NO: 15 - protein, 7 aa, Artificial Sequence
  Description of Artificial Sequence Synthetic peptide
KHATVLK

SEQ ID NO: 16 - protein, 10 aa, Artificial Sequence
  Description of Artificial Sequence Synthetic peptide
KGMALLHRLK

SEQ ID NO: 17 - protein, 20 aa, Artificial Sequence
  Description of Artificial Sequence Synthetic peptide
HHHHHHVDDDDKENLYFQSK

SEQ ID NO: 18 - protein, 7 aa, Artificial Sequence
  Description of Artificial Sequence Synthetic peptide
KPSQAHK

SEQ ID NO: 19 - protein, 11 aa, Artificial Sequence
  Description of Artificial Sequence Synthetic peptide
KQTYTGIHLDK

SEQ ID NO: 20 - protein, 10 aa, Artificial Sequence
  Description of Artificial Sequence Synthetic peptide
KPTAPHVRNK

SEQ ID NO: 21 - protein, 10 aa, Artificial Sequence
  Description of Artificial Sequence Synthetic peptide
KAALGSLAKH

SEQ ID NO: 22 - protein, 14 aa, Artificial Sequence
  Description of Artificial Sequence Synthetic peptide
KGFLCDSNFCKKDH

SEQ ID NO: 23 - protein, 10 aa, Artificial Sequence
  Description of Artificial Sequence Synthetic peptide
KKILMSFHQK

SEQ ID NO: 24 - protein, 9 aa, Artificial Sequence
  Description of Artificial Sequence Synthetic peptide
KILMSFHQK

SEQ ID NO: 25 - protein, 12 aa, Artificial Sequence
  Description of Artificial Sequence Synthetic peptide
HPKLRPILLPNK

SEQ ID NO: 26 - protein, 11 aa, Artificial Sequence
  Description of Artificial Sequence Synthetic peptide
HKGMALLHRLK

SEQ ID NO: 27 - protein, 11 aa, Artificial Sequence
  Description of Artificial Sequence Synthetic peptide
KFSLLHESTLK

SEQ ID NO: 28 - protein, 7 aa, Artificial Sequence
  Description of Artificial Sequence Synthetic peptide
KHIYRLK

SEQ ID NO: 29 - protein, 13 aa, Artificial Sequence
  Description of Artificial Sequence Synthetic peptide
HIYRLKYDVTVTK

SEQ ID NO: 30 - protein, 11 aa, Artificial Sequence
  Description of Artificial Sequence Synthetic peptide
KHNGTWQAEFK

SEQ ID NO: 31 - protein, 11 aa, Artificial Sequence
  Description of Artificial Sequence Synthetic peptide
KFFLIVQALKH

SEQ ID NO: 32 - protein, 10 aa, Artificial Sequence
  Description of Artificial Sequence Synthetic peptide
KSQDHNSASK

SEQ ID NO: 33 - protein, 12 aa, Artificial Sequence
  Description of Artificial Sequence Synthetic peptide
KKPGPAKFSLLH

SEQ ID NO: 34 - protein, 11 aa, Artificial Sequence
  Description of Artificial Sequence Synthetic peptide
KIEEILSKIYH

SEQ ID NO: 35 - protein, 7 aa, Artificial Sequence
  Description of Artificial Sequence Synthetic peptide
KVHEEMK

SEQ ID NO: 36 - protein, 8 aa, Artificial Sequence
  Description of Artificial Sequence Synthetic peptide
KAHSDDEK

SEQ ID NO: 37 - protein, 9 aa, Artificial Sequence
  Description of Artificial Sequence Synthetic peptide
HPKESRSNK

SEQ ID NO: 38 - protein, 9 aa, Artificial Sequence
  Description of Artificial Sequence Synthetic peptide
KSRSAHARK

SEQ ID NO: 39 - protein, 8 aa, Artificial Sequence
  Description of Artificial Sequence Synthetic peptide
KLYEAVHK

SEQ ID NO: 40 - protein, 11 aa, Artificial Sequence
  Description of Artificial Sequence Synthetic peptide
KNNGTWQAEFK

SEQ ID NO: 41 - protein, 11 aa, Zaire ebolavirus
KHNGTWQAEFK

(19) United States
(12) Patent Application Publication
(10) Pub. No.: US 2016/0114022 A1
(43) Pub. Date: Apr. 28, 2016

(54) THERAPIES, VACCINES, AND PREDICTIVE METHODS FOR FILOVIRUSES INCLUDING EBOLAVIRUS AND MARBURG VIRUS

(71) Applicants: Samuel Bogoch, New York, NY (US); Elenore S. Bogoch, New York, NY (US); Anne-Elenore Bogoch Borsanyi, New York, NY (US); Samuel Winston Bogoch, Oakland, CA (US)

(72) Inventors: Samuel Bogoch, New York, NY (US); Elenore S. Bogoch, New York, NY (US); Anne-Elenore Bogoch Borsanyi, New York, NY (US); Samuel Winston Bogoch, Oakland, CA (US)

(21) Appl. No.: 14/880,210

(22) Filed: Oct. 10, 2015

Related U.S. Application Data
(60) Provisional application No. 62/062,859, filed on Oct. 11, 2014.

(51) Int. Cl. A61K 39/12; C07K 16/10

(52) U.S. Cl. CPC A61K 39/12; C07K 16/10; C12N 2760/14034

(57) ABSTRACT

The present invention provides therapies, vaccines, and predictive methods for Filoviruses, including Ebolaviruses and Marburg viruses, and provides compounds for diagnosing, preventing, and treating outbreaks of Filoviruses.

This application claims priority to U.S. Provisional Application Ser. No. 62/062,859, filed Oct. 11, 2014.

### SEQUENCE LISTING

The instant application contains a Sequence Listing, which has been submitted electronically in ASCII format and is hereby incorporated by reference in its entirety. Said ASCII copy, created on Oct. 9, 2015, is named 13794-49202_SL.txt and is 10,290 bytes in size.

### FIELD OF THE INVENTION

The present invention relates to therapies for preventing and treating Filoviruses, including Ebolaviruses and Marburg viruses, methods of differentiating lethality of Filoviruses and of predicting outbreaks of lethal Filovirus, and compounds for diagnostic, therapeutic, and/or preventive purposes in Filovirus.

### BACKGROUND OF THE INVENTION

The virus family Filoviridae contains viruses known as Filoviruses. These viruses form filamentous virions and have a single-stranded negative-sense RNA genome. Ebolaviruses and Marburg viruses are Filoviruses. They cause severe disease in humans and nonhuman primates in the form of viral hemorrhagic fevers

There are currently understood to be five viruses that cause Ebola virus disease. Four of these Ebolaviruses (Bundibugyo virus, Sudan virus, Tai Forest, and Ebola virus—earlier known as Zaire Ebola virus) are understood to cause disease in humans. The fifth Ebolavirus (Reston virus) is currently thought not to cause disease in humans. Each of these five viruses is classified in the genus Ebolavirus, in the Filoviridae family, and Mononegavirales order. These viruses are understood to be closely related to Marburg viruses, which are likewise Filoviruses.

The Ebolavirus and Marburg virus genomes are understood to include seven structural proteins: polymerase cofactor (VP35 and VP 40); RNA polymerase; nucleoprotein (NP); glycoprotein (GP); transcription activator (VP30); and VP24. The genome is an approximately 19,000 nucleotide single-stranded RNA. This genome is carried within virions with a tubular appearance. The virion has nucleocapsid, matrix, and viral envelope components.

Genomes of at least two different Marburg viruses have been identified, MARV and RAVV.

Transmission of Filoviruses such as Ebolaviruses and Marburg viruses are understood to be through body fluids. Ebolaviruses are understood to be present in reservoirs in bats (such as fruit bats).

There is a need in the art for quantitative methods of predicting further progression and additional outbreaks or waning of Filoviruses, including Ebolaviruses and Marburg viruses. There is likewise a need in the art for methods of preventing, and treating Filovirus infections and outbreaks and a need for therapies against Filovirus including prophylactic therapies, such as vaccines, and treatments, such as therapies for providing passive immunity and other methods of blocking progression or transmission of Filoviruses, such as Ebolaviruses and Marburg viruses and related viruses.

Replikin peptides are a family of small peptides that have been correlated with the phenomenon of rapid replication in SARS, influenza, malaria, West Nile virus, foot and mouth disease, and many other pathogens. See, e.g., WO 2008/143717. Replikin peptides have likewise been generally correlated with the phenomenon of rapid replication in viruses, organisms, and malignancies.

Identification of Replikin peptides has provided targets for detection and treatment of pathogens, including vaccine development against virulent pathogens such as influenza virus, malaria, West Nile virus, and foot and mouth disease virus. See, e.g., WO 2008/143717. In general, knowledge of and identification of this family of peptides enables development of effective therapies and vaccines for pathogens that harbor Replikins. The phenomenon of the association of Replikins with rapid replication and virulence has been fully described in U.S. Pat. No. 7,189,800; U.S. Pat. No. 7,176,275; U.S. Pat. No. 7,442,761; U.S. Pat. No. 7,894,999, U.S. Pat. No. 8,050,871, and US 2009/0041795. Both Replikin concentration (number of Replikins per 100 amino acids) and Replikin composition have been correlated with the functional phenomenon of rapid replication.

There is a continuing need for monitoring Replikin sequences in Filoviruses to identify compounds for therapies that target Filoviruses. There is also a need to develop Replikin-based therapies that are effective across strains and within strains as they mutate over time.

In response to these continuing needs and despite extensive efforts in the art to understand infectivity and lethality in Filoviruses, including Ebolavirus and Marburg virus, and to track and predict outbreaks of Filovirus, applicants have now surprisingly applied their previous discovery of Replikin chemistry in the virus genome structure to methods of predicting outbreaks of Filovirus. They have likewise now surprisingly provided methods of identifying conserved targets in emerging of Filoviruses against which pharmaceutical compositions and vaccines are provided and likewise may be provided prior to or at the outset of any further outbreak. Such vaccine development can be undertaken in as few as seven days.

### SUMMARY OF THE INVENTION

The present invention provides compounds for diagnostic, therapeutic, and/or preventive purposes against Filoviruses, including Ebolaviruses and Marburg viruses and methods of predicting outbreaks of Filoviruses, such as Ebolaviruses and Marburg viruses.

A first non-limiting aspect of the present invention provides a composition comprising at least one Replikin sequence or homologue of a Replikin sequence from a Filovirus.

A non-limiting embodiment of the first aspect of the invention provides a composition comprising at least one peptide consisting of no more than 50 amino acid residues comprising a Replikin sequence from a Filovirus, including but not limited to an Ebolavirus or a Marburg virus, or a homologue sharing at least 50%, 60%, 70%, 80%, 90%, or 95% or more homology with a Replikin sequence from a Filovirus, including but not limited to an Ebolavirus or a Marburg virus. In a non-limiting embodiment, the composition is a pharmaceutical composition. In a non-limiting embodiment, the pharmaceutical composition is a blocking composition. In a non-limiting embodiment, the pharmaceutical composition is an immunogenic composition. In a non-limiting embodiment, the pharmaceutical composition further comprises a pharmaceutically-acceptable carrier, excipient, and/or adjuvant. In a non-limiting embodiment, the Ebolavirus is Ebola Reston virus, Ebola Sudan virus, Ebola Guinea virus, Ebola Sierra (Zaire) virus, Bundibugyo virus, Tai Forest virus, or any Ebola-like virus. In a non-limiting embodiment, the Filovirus is a Marburg virus. In a non-limiting embodiment, the composition does not include SEQ ID NO: 14.

In a non-limiting embodiment of the first aspect of the invention, the composition comprises at least one peptide consisting of no more than 50 amino acid residues comprising at least one sequence of any one of SEQ ID NO(s): 1-41 or at least one homologue sharing at least 50%, 60%, 70%, 80%, 90%, or 95% or more homology with any one of SEQ ID NO(s): 1-41. In a non-limiting embodiment, the composition comprises at least one peptide consisting essentially of any one of SEQ ID NO(s): 1-41. In a non-limiting embodiment, the composition comprises at least one peptide consisting of any one of SEQ ID NO(s): 1-41. In a non-limiting embodiment, the composition comprises at least one peptide consisting of a homologue of any one of SEQ ID NO(s): 1-41, wherein said homologue shares at least 50%, 60%, 70%, 80%, 90%, or 95% or more homology with any one of SEQ ID NO(s): 1-41. In a non-limiting embodiment, a composition may comprise at least one peptide consisting of no more than 50 amino acid residues comprising at least one sequence of any one of SEQ ID NO(s): 1-13 and 15-41 or at least one homologue sharing at least 70%, 80%, 90%, or 95% or more homology with any one of SEQ ID NO(s): 1-13 and 15-41. In a non-limiting embodiment, a composition may comprise at least one peptide consisting of no more than 50 amino acid residues comprising at least one sequence of any one of SEQ ID NO(s): 2, 4, 7, 9, 15, 18, 19, 20, 26, 28, 31, or 35-41 or at least one homologue sharing at least 70%, 80%, 90%, or 95% or more homology with any one of SEQ ID NO(s): 2, 4, 7, 9, 15, 18, 19, 20, 26, 28, 31, or 35-41.

A non-limiting embodiment of the first aspect of the invention provides a pharmaceutical composition administrable to a subject susceptible to or suffering from a Filovirus infection such as Ebolavirus infection and/or a Marburg virus infection, said composition comprising at least one peptide consisting of no more than 50 amino acid residues comprising a sequence of any one of SEQ ID NO(s): 1-41 and a pharmaceutically-acceptable carrier or adjuvant.

In a non-limiting embodiment, the pharmaceutical composition comprises at least one peptide consisting of no more than 50 amino acid residues comprising a sequence of any one of SEQ ID NO(s): 2, 4, 7, 9, 15, 18, 19, 20, 26, 28, 31, or 35-41. In non-limiting embodiment, the pharmaceutical composition comprises at least one peptide consisting essentially of at least one of SEQ ID NO(s): 1-41. In a non-limiting embodiment, the pharmaceutical composition comprises at least one peptide consisting essentially of at least one of SEQ ID NO(s): 2, 4, 7, 9, 15, 18, 19, 20, 26, 28, 31, or 35-41. In a non-limiting embodiment, the pharmaceutical composition comprises at least one peptide consisting of at least one of SEQ ID NO(s): 2, 4, 7, 9, 15, 18, 19, 20, 26, 28, 31, or 35-41.

A non-limiting embodiment of the first aspect of the invention provides a pharmaceutical composition comprising a mixture of at least two peptides each consisting of no more than 50 amino acid residues, wherein each of said two peptide comprises at least one amino acid sequence of SEQ ID NO(s): 1-41 that is different from the other of said two peptides. In a non-limiting embodiment, each of said two peptides comprise at least one amino acid sequence of SEQ ID NO(s): 2, 4, 7, 9, 15, 18, 19, 20, 26, 28, 31, or 35-41 that is different from the other of said two peptides. In a non-limiting embodiment, each of said two peptides comprise at least one amino acid sequence of SEQ ID NO(s): 2, 4, 7, 9, 15, 18, 19, 20, 26, 28, 31, or 35-40 that is different from the other of said two peptides. In a non-limiting embodiment, each of said two peptides consist of at least one amino acid sequence of SEQ ID NO(s): 2, 4, 7, 9, 15, 18, 19, 20, 26, 28, 31, 35, or 39 that is different from the other of said two peptides.

In a non-limiting embodiment, a pharmaceutical composition comprises a mixture of at least three, four, five, six, seven, eight, nine, ten, eleven, twelve, thirteen, fourteen, fifteen, sixteen, seventeen, eighteen, nineteen, twenty or more different peptides of SEQ ID NO(s): 1-41.

A non-limiting embodiment of the first aspect of the invention provides a pharmaceutical composition comprising a mixture of at least thirteen peptides consisting of no more than 50 amino acid residues, wherein each of said at least thirteen peptides comprises at least one amino acid sequence of SEQ ID NO(s): 2, 4, 7, 9, 15, 18, 19, 20, 26, 28, 31, 35, and 39 that is different from the sequence of each of the other at least thirteen peptides. In a non-limiting embodiment, each of said at least thirteen peptides consists of a different sequence of SEQ ID NO(s): 2, 4, 7, 9, 15, 18, 19, 20, 26, 28, 31, 35, and 39.

In a non-limiting embodiment, a pharmaceutical composition further comprises four additional peptides, wherein each of said four additional peptides consists of a different one of SEQ ID NO(s): 36, 37, 38, and 40.

In a non-limiting embodiment of the first aspect of the invention, administration of a pharmaceutical composition to a subject having an immune system stimulates an immune response against any one of the amino acid sequences of SEQ ID NO(s): 1-41.

A non-limiting second aspect of the present invention provides a vaccine against a Filovirus. In a non-limiting embodiment, the vaccine is against an Ebolavirus or Marburg virus. In a non-limiting embodiment, the vaccine comprises a pharmaceutical composition of the first aspect of the invention.

In a non-limiting embodiment, the vaccine is against any Ebola-like virus. In a non-limiting embodiment, the vaccine is against Ebolavirus or Marburg virus. In a non-limiting embodiment, the vaccine is against Ebola Reston virus, Ebola Sudan virus, Ebola Guinea virus, Ebola Sierra (Zaire) virus, Bundibugyo virus, Tai Forest virus, or any Ebola-like virus. In a non-limiting embodiment, the vaccine is against Marburg virus. In a non-limiting embodiment, the Marburg virus is MARV or RAVV.

A non-limiting third aspect of the present invention provides a method of preventing or treating a Filovirus infection comprising administering a pharmaceutical composition of the first non-limiting aspect of the invention or a vaccine of the second non-limiting aspect of the invention to a subject susceptible to or suffering from a Filovirus infection. In a non-limiting embodiment, administration of the pharmaceutical composition or vaccine stimulates an immune response in the subject against a Filovirus. In a non-limiting embodiment, administration of the pharmaceutical composition or vaccine provides a blocking effect against a Filovirus. In a non-limiting embodiment, the Filovirus infection is an Ebolavirus infection of Marburg virus infection.

A non-limiting fourth aspect of the present invention provides a method of stimulating the immune system of a subject against a Filovirus comprising administering a pharmaceutical composition of the first aspect of the invention or a vaccine of the second aspect of the invention to the subject. In a non-limiting embodiment, the subject is suitable for providing antibodies against a Filovirus. In a non-limiting embodiment, the Filovirus is Ebolavirus and/or Marburg. In a non-limiting embodiment, the subject is suitable for production of polyclonal antibodies. In a non-limiting embodiment, the subject is suitable for production of monoclonal antibodies. In a non-limiting embodiment, the polyclonal antibodies or monoclonal antibodies are useful for providing passive immunity in a different subject. In a non-limiting embodiment, the polyclonal antibodies or monoclonal antibodies are useful for identifying Ebolavirus or Marburg virus and/or are useful for diagnosing Ebolavirus or Marburg virus.

A non-limiting fifth aspect of the present invention provides an isolated, chemically-synthesized, or recombinantly-generated binding molecule that specifically binds a Replikin sequence of a Filovirus and/or that specifically binds a homologue of a Replikin sequence of a Filovirus, wherein the homologue is at least 50%, 605, 70%, 80%, 90%, 95%, or more homologous with a Replikin sequence of a Filovirus. In a non-limiting embodiment, the Replikin sequence is not SEQ ID NO: 14. In a non-limiting embodiment, the Filovirus is Ebolavirus or Marburg virus.

In a non-limiting embodiment of the fifth aspect of the present invention, the binding molecule binds at least one sequence of SEQ ID NO(s): 1-41 or a homologue of at least one sequence of SEQ ID NO(s): 1-41, wherein said homologue is at least 50%, 60%, 70%, 80%, 90%, 95%, or more homologous. In a non-limiting embodiment the binding molecule specifically binds at least one sequence of SEQ ID NO(s): 2, 4, 7, 9, 15, 18, 19, 20, 26, 28, 31, and 35-41.

In a non-limiting embodiment, the binding molecule is an antibody or antibody fragment. In a non-limiting embodiment, the antibody is a monoclonal antibody. In a non-limiting embodiment, the antibody is a humanized antibody. In a non-limiting embodiment, the antibody is an optimized antibody. In a non-limiting embodiment, the antibody is an fc-optimized antibody. In a non-limiting embodiment, the antibody is selected using phage display. In a non-limiting embodiment, the antibody is produced from a microorganism such as E. coli.

A non-limiting sixth aspect of the present invention provides methods of delivering passive immunity to a patient suffering from a Filovirus infection comprising administering to the patient at least one isolated, chemically-synthesized, or recombinantly-generated binding molecule of the fifth aspect of the invention. In a non-limiting embodiment, the Filovirus infection is an Ebolavirus infection or a Marburg virus infection.

A non-limiting seventh aspect of the present invention provides a method of making a pharmaceutical composition comprising: selecting at least one isolated or synthesized protein, protein fragment, polypeptide, or peptide comprising at least one peptide sequence that is at least 30%, 40%, 50%, 60%, 70%, 80%, 90% or 95%, or 100% homologous with at least one Replikin peptide sequence identified in a Filovirus as a component of a pharmaceutical composition; and making said pharmaceutical composition. In a non-limiting embodiment, the method of making a pharmaceutical composition comprises selecting at least one isolated or synthesized peptide of SEQ ID NO(s): 1-13 and 15-41, as at least one component, and making said vaccine with the at least one component. In a non-limiting embodiment, the isolated or synthesized peptide comprises any one of SEQ ID NO(s): 1-13 and 15-41 and is up to 50, 60, 70, 80, 90, 100, 150, 200, or 250 residues in length. In a non-limiting embodiment, the sequence is not SEQ ID NO: 14 and/or is not a sequence from polymerase protein or RNA polymerase protein. In a non-limiting embodiment, the pharmaceutical composition is comprised in a vaccine.

In another non-limiting embodiment, a method of making a pharmaceutical composition comprises selecting at least two, three, four, five, six, seven, eight, nine, ten, eleven, twelve, thirteen, fourteen, fifteen, sixteen, seventeen, or twenty or more isolated or synthesized Replikin peptide sequences identified in Filovirus and/or isolated or synthesized functional fragments of Replikin peptide sequences identified in Filovirus and/or isolated or synthesized homologues of Replikin peptide sequences identified in Filovirus and making a pharmaceutical composition comprising said selected peptide sequences or functional fragments thereof. In a further embodiment, the isolated or synthesized Replikin peptide sequences, homologs, or functional fragments thereof comprise at least one peptide sequence of SEQ ID NO(s): 1-13 and 15-41, at least one functional fragment of at least one peptide sequence of SEQ ID NO(s): 1-13 and 15-41, or at least one functional fragment of at least one Replikin peptide sequence identified in a Filovirus and may consist of said sequences and may comprise said sequences and be up to 50, 60, 70, 80, 90, 100, 150, 200, 250 or more residues in length. In another non-limiting embodiment, the at least one isolated or synthesized protein, protein fragment, polypeptide, or peptide has the same amino acid sequence as at least one protein, protein fragment, polypeptide or peptide identified in a relatively lethal strain of Filovirus up to seven days, one month, six months, one year, two years, or three years prior to making said vaccine.

An eighth non-limiting aspect of the present invention provides a method of determining an increased probability of an outbreak of Filovirus within about one year following an increase in Replikin concentration in an isolate of Filovirus comprising identifying an increase in the concentration of Replikin sequences in at least one first isolate of Filovirus as compared to at least one other isolate of Filovirus wherein said at least one first isolate is isolated at a later time period than said one other isolate and wherein said increase in the concentration of Replikin sequences signifies the increased probability of the outbreak of Filovirus within about one year following the increase in the concentration of Replikin sequences. In a non-limiting embodiment, the Filovirus is an Ebolavirus or a Marburg virus.

In a non-limiting embodiment, a method of prediction comprises: (1) obtaining a plurality of isolates of a Filovirus wherein at least one of said isolates is isolated about six months to about 3 years later than at least one other of said isolates; (2) analyzing the amino acid sequence of at least one protein or protein fragment in each isolate of the plurality of isolates for the presence and concentration of Replikin sequences; (3) comparing the concentrations of Replikin sequences in the at least one protein or protein fragment in each isolate of the plurality of isolates one to another; (4) identifying an increase in the concentration of Replikin sequences in said plurality of isolates over at least one time period of about six months or greater; and (5) predicting an outbreak of the Filovirus within about one month to about three years following said identified increase in the concentration of Replikin sequences. In another embodiment of the invention, the Filovirus outbreak is predicted within about six months. In a further embodiment, the Filovirus outbreak is predicted within about one year to about three years. In a further non-limiting embodiment, the method of prediction further comprises processing at least one step of the method on a computer. In a non-limiting embodiment, the Filovirus may be an Ebolavirus or Marburg virus.

In a further non-limiting embodiment, the protein or protein fragment is from VP35, polymerase, or glycoprotein protein of a Filovirus. A Filovirus may include, but is not limited to an Ebolavirus or Marburg virus.

An ninth non-limiting aspect of the present invention provides a nucleic acid sequence that is antisense to a nucleic acid that encodes for any Replikin peptide present in or identified in an isolate Filovirus. This may include one of SEQ ID NO(s): 1-35, 39, or 41 or a small interfering nucleic acid sequence that interferes with a nucleic acid sequence that is 30%, 40%, 50%, 60%, 70%, 80%, 90%, or 95% or more homologous with a nucleic acid that encodes any Replikin peptide of a Filovirus including, for example, any one of SEQ ID NO(s): 1-35, 39, or 41 or is 30%, 40%, 50%, 60%, 70%, 80%, 90%, 95% or more homologous with a nucleic acid that is antisense to a nucleic acid that encodes for any one of SEQ ID NO(s): 1-35, 39, or 41. In a non-limiting embodiment, the nucleic acid sequence is 21 to 150 nucleotides in length. In a non-limiting embodiment, the nucleic acid sequence is up to 5, 10, 15, 20, 25, 30, 35, 40, 45, or 50 nucleotides in length. In a non-limiting embodiment, the nucleic acid encodes for or is antisense to a nucleic acid that encodes for a homologue of a Replikin peptide of Filovirus. In a non-limiting embodiment, the nucleic acid encodes for or is antisense to a nucleic acid that encodes for any one of SEQ ID NO(s): 36, 37, 38, or 40.

### BRIEF DESCRIPTION OF THE DRAWINGS

FIG. 1 illustrates the annual mean Replikin concentration of all Ebola sequences from animal and human subjects available to applicants in a given year when queried at the NCBI PubMed database website for isolates from 1976 through September 2014. 10,197 sequences were available in the database when queried. 5,820 sequences were observed to contain a Replikin sequence. Dark lines reflect mean Replikin concentration. Lighter lines reflect standard deviation from the annual mean. A peak in 2013 correlates with the 2014 high-virulence, high-lethality outbreak of Ebola in 2014.

FIG. 2 illustrates the annual mean Replikin concentration of all glycoprotein Ebola sequences from animal and human subjects available to applicants in a given year when queried at the NCBI PubMed database website for isolates from 1976 through September 2014. 777 sequences were available in the database when queried. Dark lines reflect mean Replikin concentration. Lighter lines reflect standard deviation from the annual mean. A peak in 2013 correlates with the 2014 high-virulence, high-lethality outbreak of Ebola in 2014.

FIG. 3 illustrates the annual mean Replikin concentration of all VP35 Ebola sequences from animal and human subjects available to applicants in a given year when queried at the NCBI PubMed database website for isolates from 2004 through September 2014. 747 sequences were available in the database when queried. Dark lines reflect mean Replikin concentration. Lighter lines reflect standard deviation from the annual mean. A peak in 2012 correlates with and predicts the 2014 high-virulence, high-lethality outbreak of Ebola two years later in 2014.

FIG. 4 illustrates the annual mean Replikin concentration of all polymerase Ebola sequences from animal and human subjects available to applicants when queried at the NCBI PubMed database website for isolates from 2004 through September 2014. 782 sequences were available in the database when queried. Dark lines reflect mean Replikin concentration. Lighter lines reflect standard deviation from the annual mean. A peak in 2012 correlates with and predicts the 2014 high-virulence, high-lethality outbreak of Ebola two years later in 2014.

FIG. 5 illustrates the annual mean Replikin concentration of all Marburg Ebola sequences from animal and human subjects available to applicants in a given year when queried at the NCBI PubMed database website for isolates from 1976 through September 2014. 652 sequences were available in the database when queried. Dark lines reflect mean Replikin concentration. Lighter lines reflect standard deviation from the annual mean. A peak in 2013 correlates with the 2014 high-virulence, high-lethality outbreak of Ebola in 2014.

FIG. 6 illustrates an increase in Replikin concentration (Replikin sequences per 100 amino acid residues) in spike and nucleocapsid coronavirus proteins preceding the SARS coronavirus epidemic of 2003. The x-axis indicates the year and the y-axis indicates the Replikin concentration. The appearance of the SARS outbreak and the eight countries involved in the outbreak is shown by the conical shaded area. The solid black symbols represent the mean Replikin concentration for spike coronavirus proteins and the vertical black bars represent the standard deviation of the mean. Replikin concentration rose between 1995 and 2002, consistent with the SARS coronavirus outbreak, which emerged at the end of 2002 and persisted into 2003. The decline in Replikin concentration correctly signaled the end of the SARS outbreak and had already begun its return to pre-outbreak levels when the outbreak emerged.

FIG. 7 illustrates the annual mean Replikin concentration of all Marburg Ebola sequences from animal and human subjects available to applicants in a given year when queried at the NCBI PubMed database website for isolates from 1976 through September 2015. Dark lines reflect mean Replikin concentration. Lighter lines reflect standard deviation from the annual mean. A peak in 2013 correlates with the 2014 high-virulence, high-lethality outbreak of Ebola in 2014.

FIG. 8 illustrates observation of a strong immune response in a rabbit inoculated with a peptide composition. The Y-axis reflects mean optical density of various observations of triplicate ELISA tests of rabbit blood drawn at various times before, during, and after the vaccination regimen. The X-axis reflects the various bleeds taken during the regimen. Pre-bleed was drawn and tested at day 0. 1^{st} bleed was drawn and tested at day 22. 2^{nd} bleed was drawn and tested on day 36. 3^{rd} bleed was drawn and tested on day 57. 4^{th} bleed was drawn and tested on day 87. FIG. 8 illustrates a strong immune response over the 87-day period.

### DETAILED DESCRIPTION OF THE INVENTION

### Definitions

A “protein fragment” as used in this specification is any fragment of an expressed whole protein, which is any portion of an expressed whole protein where a “portion” of a protein is less than an expressed whole protein. A protein fragment is not naturally produced as part of the biological function of the virus. A protein fragment reflects an expressed whole protein with one or more amino acids removed from the amino acid sequence of the expressed whole protein. A protein fragment may also reflect an amino acid sequence that is at least 30%, 40%, 50%, 60%, 70%, 80%, 90%, 95%, or more homologous with any portion of an expressed whole protein so long as the protein fragment does not reflect the entire amino acid sequence of the expressed whole protein or the functional protein following biological processing. A “polypeptide,” as used in this specification, is any portion of a protein fragment and is less than an expressed whole protein. A protein fragment may reflect 50, 100, 150, 200, or more amino acid residues (or any size in between) so long as the protein fragment reflects less than the total length of the expressed whole protein.

A “functional fragment” of a Replikin sequence as described herein is a fragment, variant, analog, or chemical derivative of a Replikin sequence that retains at least a portion of the immunological cross reactivity with an antibody specific for the Replikin sequence. A fragment of the Replikin sequence refers to any subset of the molecule. Variant peptides of the sequence may be made by direct chemical synthesis, for example, using methods well known in the art. An analog of a Replikin sequence to a non-natural protein or polypeptide is substantially similar to either the Replikin sequence of the protein or a fragment thereof. Chemical derivatives of a Replikin sequence contain additional chemical moieties. Amino acid analogues may include D-amino acid residues, or polypeptides with modified structural backbones, or polypeptides with non-natural chemical moieties added.

As used herein, the term “preferentially binds” or “specifically binds” and related terms referencing the interaction of a binding molecule such as, for example, an antibody, and the structure to which it binds (antigen) means that the binding molecule preferentially recognizes the structure to which it binds even when present among other molecules (such as in a mixture of molecules). Specific or preferential binding of a binding molecule to a binding structure or an immunogenic portion of a binding structure is specific and preferential when the binding molecule binds to the structure or portion thereof and does not bind with the same level of affinity to other structures. Binding affinity may be determined by one of ordinary skill in the art using, for example, BIACORE, enzyme-linked immunosorbent assays, or radioimmuno assays. A binding molecule may cross-react with related antigens and preferably does not cross-react with affinity to unrelated antigens. Binding between a binding molecule and the structure to which it binds may be mediated by covalent or non-covalent attachment, or both.

As used herein a “vaccine” is any substance, compound, composition, mixture, or other therapeutic substance that, when administered to a human or animal via any method of administration known to the skilled artisan now or hereafter, produces an immune response, an antibody response, or a protective effect in the human or animal.

As used herein, a “Replikin sequence” is an amino acid sequence of 7 to 50 amino acid residues comprising (1) a first lysine residue located six to ten residues from a second lysine residue; (2) at least one histidine residue; and (3) at least 6% lysine residues, where the sequence is the shortest sequence comprising the first and second lysine residues of element (1) and the at least one histidine of element (2). A Replikin sequence may comprise more than two lysine residues and more than one histidine residue so long as at least two of the lysine residues and at least one histidine residue reflect the requirements of the definition of a Replikin sequence. For diagnostic, therapeutic, and preventive purposes, a Replikin sequence may or may not be the shortest sequence comprising the first and second lysine residues of element (1) and the at least one histidine residue of element (2). A Replikin sequence may comprise a terminal lysine residue and a terminal lysine or histidine residue where the sequence is 7 to 50 amino acid residues in length and comprises (1) at least one lysine residue located six to ten residues from at least one other lysine residue; (2) at least one histidine residue; and (3) at least 6% lysine residues.

The term “Replikin sequence” can also refer to a nucleic acid sequence encoding an amino acid sequence having 7 to about 50 amino acids comprising:
(1) at least one lysine residue located six to ten amino acid residues from a second lysine residue;
(2) at least one histidine residue; and
(3) at least 6% lysine residues.

As used herein, an “isolated” peptide may be synthesized by organic chemical methods. An isolated peptide may also be synthesized by biosynthetic methods. An isolated peptide may also refer to a peptide that is, after purification, substantially free of cellular material or other contaminating proteins or peptides from the cell or tissue source from which the peptide is derived, or substantially free from chemical precursors or other chemicals when chemically synthesized by any method, or substantially free from contaminating peptides when synthesized by recombinant gene techniques or a protein or peptide that has been isolated in silico from nucleic acid or amino acid sequences that are available through public or private databases or sequence collections. An isolated peptide may be synthesized by biosynthetic or organic chemical methods.

Proteins, protein fragments, polypeptides, or peptides in this specification may be chemically synthesized by any method known to one of skill in the art now and hereafter. For example, isolated proteins, protein fragments, polypeptides, or peptides may be synthesized by solid phase synthesis. The production of these materials by chemical synthesis avoids the inclusion of (or the need to remove by purification) materials that are byproducts of other production methods such as recombinant expression or isolation from biological material. Such byproducts may include, for example, avian proteins associated with vaccines produced using birds' eggs, bacterial proteins associated with recombinant production in bacteria, or proteins or contaminants associated with any recombinant activity such as with productions of proteins or other sequences in insect cells.

An “encoded” or “expressed” protein, protein sequence, protein fragment sequence, or peptide sequence is a sequence encoded by a nucleic acid sequence that encodes the amino acids of the protein or peptide sequence with any codon known to one of ordinary skill in the art now or hereafter. It should be noted that it is well known in the art that, due to redundancy in the genetic code, individual nucleotides can be readily exchanged in a codon and still result in an identical amino acid sequence. As will be understood by one of ordinary skill in the art, a method of identifying a Replikin amino acid sequence also encompasses a method of identifying a nucleic acid sequence that encodes a Replikin amino acid sequence wherein the Replikin amino acid sequence is encoded by the identified nucleic acid sequence.

“Homologous” or “homology” or “sequence identity” as used in this specification indicate that an amino acid sequence or nucleic acid sequence exhibits substantial structural equivalence with another sequence, namely any Replikin peptide sequence (including SEQ ID NO(s): 1-35) identified in an isolate of Ebolavirus or any nucleotide sequence encoding a Replikin peptide sequence in an isolate of Ebolavirus (a redundancy in a coding sequence may be considered identical to a sequence encoding the same amino acid). To determine the percent identity or percent homology of an identified sequence, a sequence is aligned for optimal comparison purposes with any one of possible basis sequences. For purposes of this paragraph, a basis sequence is a Replikin sequence identified in an isolate of Ebolavirus. Where gaps are necessary to provide optimal alignment, gaps may be introduced in the identified sequence or in the basis sequence. When a position in the identified sequence is occupied by the same amino acid residue or same nucleotide as the corresponding position in the basis sequence, the molecules are considered identical at that position (as used herein amino acid or nucleic acid “identity” is equivalent to amino acid or nucleic acid “homology”). To determine percent homology, the amino acid residues or nucleotides at corresponding amino acid positions or nucleotide positions are compared between the identified sequence and the basis sequence. The total number of amino acid residues or nucleotides in the identified sequence that are identical with amino acid residues or nucleotides in the basis sequence is divided by the total number of residues or nucleotides in the basis sequence (if the number of residues or nucleotides in the basis sequence is greater than the total number of residues or nucleotides in the identified sequence) or by the total number of amino acid residues or nucleotides in the identified sequence (if the number of residues or nucleotides in the identified sequence is greater than the total number of residues or nucleotides in the basis sequence). The final number is determined as a percentage. As such, the percent identity between the two sequences is a function of the number of identical positions shared by the sequences, taking into account the number of gaps (where a gap must be introduced for optimal alignment of the two sequences) and the length of each gap. Any structural or functional differences between sequences having sequence identity or homology will not affect the ability of the sequence to function as indicated in the desired application.

For example, SEQ ID NO: 15 (KHATVLK) is considered 70% homologous with SEQ ID NO: 13 (KVKKHATVLK). The 70% homology between SEQ ID NO: 15 and SEQ ID NO: 13 is determined as follows: SEQ ID NO: 15 is the identified sequence. SEQ ID NO: 13 is the basis sequence. Upon alignment, SEQ ID NO: 15 is identical to SEQ ID NO: 13 in seven of the ten sequences of SEQ ID NO: 13. To determine percent homology, then, the 7 aligned identical residues are divided by the total number of residues in SEQ ID NO: 13, namely 10 residues, giving 0.70 or more than 70% homology.

To determine homology between an identified sequence that is contained in a larger polypeptide, protein fragment, or protein, and a basis sequence, the polypeptide, protein fragment, or protein must first be optimally aligned with the basis sequence. Upon alignment of the sequences, the residue in the identified sequence that is furthest to the amino-terminus of the polypeptide, protein fragment, or protein and identical to a residue in the basis sequence that is furthest to the amino-terminus of the basis sequence is considered the amino-terminal residue of the identified sequence. Likewise, upon alignment, the residue in the identified sequence that is furthest to the carboxy-terminus of the polypeptide, protein fragment, or protein and identical to a residue in the basis sequence that is furthest to the carboxy-terminus of the basis sequence is considered the carboxy-terminal residue of the identified sequence.

Concerning gaps, the number of gaps in either the basis sequence or the identified sequence should be limited to the number of gaps allowable without significantly compromising the function of the identified sequence as compared to the basis sequence. In general, many gaps in the sequence of the basis peptide or in the sequence of the identified peptide are allowed based on homology as defined herein. Relatively more gaps are allowed if the lysines and histidines that create the definition of the Replikin peptide are identically shared between the basis peptide and the identified peptide. Relatively more gaps are also allowed if the lysines and histidines that create the definition of the Replikin peptide are shared at least in close position (for example within ten, nine, eight, seven, six, five, four, three, two, or one amino acid residue). If some of the lysine residues and histidine residues that create the definition of the Replikin peptide are not present in the identified peptide, fewer gaps may be allowed. Nevertheless, if the identified peptide functions similarly to the basis peptide, any number of gaps is allowed. In general, three or more gaps are allowed in the sequence of the basis peptide or in the sequence of the identified peptide within ten amino acid residues of the basis peptide if no lysines or histidines are present in the identified peptide. Two or more gaps or one or more gaps are also allowed. Nevertheless, if the identified sequence provides the same or a similar function to the basis sequence, more gaps are allowed up to the number of gaps that will provide a homology of 30%, 40%, 50%, 60%, 70%, 80%, 90%, 95%, or more homology. Additionally, where the lysines and histidines of the Replikin definition are present in both the identified peptide and the basis peptide, there should be no limit on how many gaps are allowed.

The presence of lysines and histidines providing for the Replikin definition in an identified peptide requires significantly less homology because the lysines and the histidines of the Replikin definition provide for conservation of Replikin function. For example, in Table 8 and the description thereof in columns 62 and 63 in U.S. Pat. No. 7,442,761, a highly mutable tat protein in HIV is described and analyzed. As may be seen from Table 8 in U.S. Pat. No. 7,442,761, in tat protein of HIV, which is essential for replication in the virus, lysines and histidines that are essential to maintaining the Replikin definition within a key Replikin peptide in the protein are observed to be 100% conserved, while substitutions in amino acid residues that are not essential to maintaining the Replikin definition are commonly substituted. The conservation of the key amino acids for maintaining the Replikin definition is understood to provide a specific survival function for HIV. The same phenomenon is seen in influenza. See U.S. Pat. No. 7,442,761, column 62, lines 42-45. This phenomenon has now also been surprisingly identified in Ebolavirus. See, e.g., Table 12.

As used herein, “Replikin Count” or “Replikin concentration” refers to the number of Replikin sequences per 100 amino acids in a protein, protein fragment, virus, or organism. A higher Replikin concentration in a first strain of a virus or organism has been found to correlate with more rapid replication of the first virus or organism as compared to a second, earlier-arising or later-arising strain of the virus or organism having a lower Replikin concentration. Replikin concentration is determined by counting the number of Replikin sequences in a given sequence, wherein a Replikin sequence is a peptide of 7 to 50 amino acid residues comprising (1) a first lysine residue six to ten residues from a second lysine residue, (2) at least one histidine residue, (3) and 6% or more lysine residues where the Replikin sequence is the shortest sequence comprising the first and second lysine residues of element (1) and the at least one histidine residue of element (2). A Replikin sequence may comprise more than two lysine residues and more than one histidine residue so long as there is at least one lysine residue six to ten residues from a second lysine residue and at least one histidine residue. A Replikin sequence for the purpose of determining Replikin concentration as described in this paragraph may also be a nucleic acid that encodes a Replikin peptide sequence defined according to this paragraph.

### Pharmaceutical Compositions

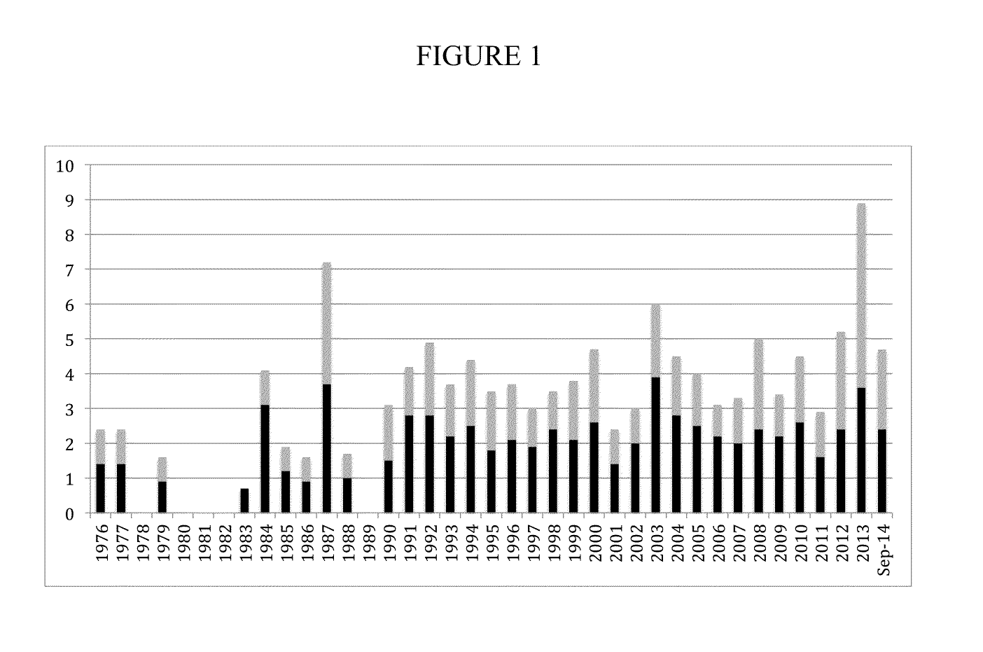

As illustrated in FIGS. 1-5 and 7, Replikin sequences in Filoviruses, including Ebolaviruses and Marburg virus are structurally and functionally related to rapid replication, virulence, lethality, and viral outbreaks. Further, Tables 12 and 13 in Example 2 demonstrate Replikin sequences are highly conserved across strains and geographic occurrences of highly-lethal Filoviruses. Identification of these structures in Filoviruses, therefore, allows for targeting of the function of rapid replication (related to virulence and lethality) in Filoviruses by stimulating an immune response against these functional targets and by administering blocking compounds (including Replikin sequences, binding molecules against Replikin sequences, and antisense nucleic acid sequences against Replikin sequences) that interfere with these functional targets.

One non-limiting aspect of the invention, therefore, provides compounds and compositions that stimulate an immune response against or otherwise mechanically block Replikin sequences resulting in inhibition of replication in the virus and diminishment of virulence and lethality. Pharmaceutical compositions that target highly-conserved sequences and/or sequences present in a current outbreak are useful for treating and preventing Ebolavirus and other Filovirus infections including, for example, Bundibugyo virus, Sudan virus, Tai Forest, Ebola virus, Reston virus, and Marburg virus.

Compounds and the active part of said compositions may be produced, for example, by solid-phase synthesis of peptide sequences. These compositions need not contain biologics and need not be refrigerated. Production may be ramped up easily with economies of scale to meet large populations (even the global population). Compositions may be manufactured in as little as seven days. Challenge studies in Ebolavirus have demonstrated a strong immune response. Other studies in influenza and taura syndrome virus have demonstrated both an immune response and blocking responses. See, e.g., US 2009/0041795 and US 2010/0215675 (each incorporated herein by reference).

A non-limiting aspect of the present invention therefore provides a composition comprising at least one peptide consisting of no more than 50 amino acid residues comprising a Replikin sequence from a Filovirus such as an Ebolavirus or a Marburg virus or a homologue sharing at least 50%, 60%, 70%, 80%, 90%, or 95% or more homology with a Replikin sequence from a Filovirus. The composition may be a pharmaceutical composition and may be a blocking composition and/or an immunogenic composition. A pharmaceutical composition may comprise a pharmaceutically-acceptable carrier, excipient, and/or adjuvant. A blocking or immune response in a subject administered a pharmaceutical composition may be directed against Ebola Reston virus, Ebola Sudan virus, Ebola Guinea virus, Ebola Sierra (Zaire) virus, Marburg virus, Bundibugyo virus, Tai Forest, Ebola virus, any Ebola-like virus, and any Filovirus. In a non-limiting embodiment, the composition does not include SEQ ID NO: 14.

A composition, including a pharmaceutical composition such as a blocking composition and/or immunogenic composition may comprise at least one peptide that consists of no more than 50 amino acid residues comprising at least one sequence of any one of SEQ ID NO(s): 1-13 and 15-41 or at least one homologue sharing at least 50%, 60%, 70%, 80%, 90%, or 95% or more homology with any one of SEQ ID NO(s): 1-13 and 15-41. A composition may comprise SEQ ID NO: 14 or a homologue thereof or a peptide of up to 50 residues comprising SEQ ID NO: 14 or a homologue thereof. A composition may comprise at least one peptide consisting essentially of any one of SEQ ID NO(s): 1-13 and 15-41. A composition may comprise at least one peptide consisting of any one of SEQ ID NO(s): 1-13 and 15-41. A composition may comprise at least one peptide consisting of a homologue of any one of SEQ ID NO(s): 1-41, wherein said homologue shares at least 50%, 60%, 70%, 80%, 90%, or 95% or more homology with any one of SEQ ID NO(s): 1-41.

A non-limiting embodiment provides a pharmaceutical composition administrable to a subject susceptible to or suffering from a Filovirus infection such as an Ebolavirus infection and/or a Marburg virus infection. The composition may comprise at least one peptide consisting of no more than 50 amino acid residues comprising a sequence of any one of SEQ ID NO(s): 1-13 and 15-41 and a pharmaceutically-acceptable carrier or adjuvant. The composition may comprise at least one peptide consisting of no more than 50 amino acid residues comprising a sequence of SEQ ID NO: 14 and a pharmaceutically-acceptable carrier or adjuvant.

A pharmaceutical composition may comprise at least one peptide consisting of no more than 50 amino acid residues comprising a sequence of any one of SEQ ID NO(s): 2, 4, 7, 9, 15, 18, 19, 20, 26, 28, 31, or 35-41. A pharmaceutical composition may comprise at least one peptide consisting essentially of at least one of SEQ ID NO(s): 2, 4, 7, 9, 15, 18, 19, 20, 26, 28, 31, or 35-41. A pharmaceutical composition may comprise at least one peptide consisting of at least one of SEQ ID NO(s): 2, 4, 7, 9, 15, 18, 19, 20, 26, 28, 31, or 35-41.

A pharmaceutical composition may comprise a mixture of Replikin sequences and/or homologues of Replikin sequences from Filovirus. For example, a pharmaceutical composition may comprise a mixture of at least two peptides, wherein each of the at least two peptide consist of no more than 50 amino acid residues and each of the at least two peptides comprise at least one amino acid sequence of SEQ ID NO(s): 1-41 that is different from the other of the at least two peptides. Each of the two peptides may comprise at least one amino acid sequence of SEQ ID NO(s): 2, 4, 7, 9, 15, 18, 19, 20, 26, 28, 31, or 35-41 that is different from the other of the at least two peptides. Each of the two peptides may comprise at least one amino acid sequence of SEQ ID NO(s): 2, 4, 7, 9, 15, 18, 19, 20, 26, 28, 31, or 35-40 that is different from the other of the at least two peptides. In a non-limiting embodiment, each of the two peptides consists of at least one amino acid sequence of SEQ ID NO(s): 2, 4, 7, 9, 15, 18, 19, 20, 26, 28, 31, 35, or 39 that is different from the other of the two peptides.

A non-limiting embodiment provides a pharmaceutical composition comprising a mixture of at least thirteen peptides, wherein each the at least thirteen peptide consists of no more than 50 amino acid residues and each of the at least thirteen peptides comprises at least one amino acid sequence of SEQ ID NO(s): 2, 4, 7, 9, 15, 18, 19, 20, 26, 28, 31, 35, and 39 that is different from each of the other at least thirteen peptides. Each of the at least thirteen peptides may consist of a different sequence of SEQ ID NO(s): 2, 4, 7, 9, 15, 18, 19, 20, 26, 28, 31, 35, and 39.

A pharmaceutical composition may further comprise four additional peptides, wherein each of said four additional peptides consists of a different one of SEQ ID NO(s): 36, 37, 38, and 40. Each of the four additional peptides may consist of up to 50 amino acid residues and comprise at least one amino acid sequence of SEQ ID NO(s): 36, 37, 38, and 40 that is different from each of the other of the four additional peptides.

Administration of a pharmaceutical composition to a subject having an immune system is understood to result in an immune response and/or blocking response against the amino acid sequences of SEQ ID NO(s): 1-41. As a result, the pharmaceutical composition is useful for targeting replication, virulence, and lethality in Filoviruses, including Ebolaviruses and Marburg viruses.

Use of a herein-described peptide sequence for the manufacture of a pharmaceutical composition against Filoviruses is provided. Use of a herein-described pharmaceutical composition for administration to an animal to provide a blocking response and/or an immune response and a resulting protective effect against Filovirus infection is provided.

### Blocking and Immunogenic Compounds

A non-limiting immunogenic and/or blocking compound is provided comprising at least one protein, protein fragment, polypeptide, or peptide of any one of the proteins, protein fragments, polypeptides, or peptides described herein including and not limited to comprising at least one Replikin peptide sequence identified in a Filovirus, including an Ebolavirus or a Marburg virus, or at least one homologue of said at least one Replikin peptide sequence, or at least one functional fragment of at least one Replikin peptide sequence. In a non-limiting embodiment, an immunogenic and/or blocking compound does not comprise SEQ ID NO: 14. In a further non-limiting embodiment, the immunogenic and/or blocking compound does not comprise a Replikin sequence identified in a polymerase protein or an RNA polymerase protein.

In a non-limiting embodiment, the immunogenic and/or blocking compound comprises at least one peptide sequence of SEQ ID NO(s): 1-13 and 15-41, consists essentially of any one of SEQ ID NO(s): 1-13 and 15-41, or consists of any one of SEQ ID NO(s): 1-13 and 15-41 or at least one functional fragment of any one of SEQ ID NO(s): 1-13 and 15-41. A Replikin sequence may be shared among different isolates of Filovirus, including isolates of Ebolaviruses and/or Marburg viruses. The sequence may be shared among any two or more of isolates of Ebola Reston virus, Ebola Sudan virus, Ebola Guinea virus, Ebola Sierra (Zaire) virus, Marburg virus, Bundibugyo virus, Taï Forest, Ebola virus, any Ebola-like virus, or any Filovirus. The Replikin sequences may be shared as identical or as close homologues. The Replikin sequences may differ by a single amino acid residue. The Replikin sequences may be conserved in Filoviruses over one, two, three, or more years.

### Preventing and Treating Filovirus Infections

Because Replikin sequences may be targeted to inhibit or control replication, virulence, and lethality in Filoviruses, a method of preventing or treating Filovirus infection, including Ebolavirus infection of Marburg virus infection, is provided comprising administering a pharmaceutical composition or a vaccine comprising a immunological and/or blocking composition to a subject susceptible to or suffering from a Filovirus infection, such as an Ebolavirus infection and/or a Marburg virus infection. Administration of a pharmaceutical composition or vaccine stimulates an immune response in the subject against Filoviruses such as Ebolavirus and/or Marburg virus. Administration of a pharmaceutical composition or vaccine provides a blocking response against Filovirus such as Ebolavirus and/or Marburg virus.

The pharmaceutical compositions of the invention, alone or in various combinations, may be administered to a subject by any manner known to one of ordinary skill in the art including by intravenous or intramuscular injection, ocular swab or spray, nasal spray and/or inhalation spray, or any other method of administration in order to stimulate the immune system of the subject to produce an immune response or in order to provide a direct or otherwise indirect blocking effect. Generally the dosage of peptides in a pharmaceutical composition is in the range of from about 0.1 μg to about 10 mg, about 10 μg to about 1 mg, and about 50 μg to about 500 μg. A non-limiting dosage may be 0.01 to 5 mg of peptide per gram of body mass of a subject. A non-limiting dosage may be 0.05 to 1 mg of peptide per gram of body mass of a subject. A non-limiting dosage may be 0.1 to 0.5 mg of peptide per gram of body mass of a subject. The skilled practitioner can readily determine the dosage and number of doses needed to produce an effective immune response or an effective blocking effect, or both.

### Stimulating Immune Responses

Replikin sequences and peptides have been shown to be highly immunogenic where antibodies produced against the sequences or peptides target replication. See, e.g., US 2003/0194414 (incorporated herein by reference). The identification herein of function Replikin sequences conserved in Filoviruses provides a method of stimulating the immune system of a subject against Filoviruses such as Ebolavirus and/or Marburg virus comprising administering a pharmaceutical composition or a vaccine to a subject. The subject may be a suitable subject for providing antibodies against Filoviruses. The subject may be suitable for stimulation and production of polyclonal antibodies or for stimulation and production of monoclonal antibodies. The polyclonal antibodies and monoclonal antibodies are useful for providing passive immunity in a patient. The polyclonal antibodies and monoclonal antibodies are likewise useful for identifying Filoviruses such as Ebolavirus or Marburg virus and/or are useful for diagnosing Filoviruses such as Ebolavirus or Marburg virus.

### Vaccines

A vaccine is provided against Filoviruses including Ebolavirus or Marburg virus. The vaccine targets Replikin structures in Filovirus thereby limiting replication, virulence, and lethality. A vaccine may comprise a Replikin peptide or homologue or function fragment of a Replikin peptide identified in a Filovirus. A vaccine may comprise a pharmaceutical composition described herein.

A vaccine may be directed against any Ebola-like virus including Filoviruses. A vaccine may be directed against Ebolavirus or Marburg virus. A vaccine may be directed against Ebola Reston virus, Ebola Sudan virus, Ebola Guinea virus, Ebola Sierra (Zaire) virus, Marburg virus, Bundibugyo virus, Taï Forest, Ebola virus, any Ebola-like virus, or any Filovirus. A vaccine may comprise a mixture of a plurality of peptide sequences or homologues of any of SEQ ID NO(s): 1-41, a mixture of a plurality of peptide sequences and/or homologues of any of SEQ ID NO(s): 1-13 and 15-41, a mixture of a plurality of peptide sequences and/or homologues of any of SEQ ID NO(s): 2, 4, 7, 9, 15, 18, 19, 20, 26, 28, 31, and 35-40, a mixture of a plurality of peptide sequences and/or homologues of any of SEQ ID NO(s): 2, 4, 7, 9, 15, 18, 19, 20, 26, 28, 31, 35, and 39, and/or a mixture of peptide sequences and/or homologues of any of SEQ ID NO(s): 19, 34, and 35. A vaccine may comprise a mixture of a plurality of peptide sequences consisting essentially of any one or more of SEQ ID NO(s): 1-41 or a mixture of a plurality of peptide sequences consisting essentially of any one or more of SEQ ID NO(s): SEQ ID NO(s): 2, 4, 7, 9, 15, 18, 19, 20, 26, 28, 31, 35, or 39 or mixture of a plurality of peptide sequences consisting essentially of SEQ ID NO(s): 19, 34, and 35. A vaccine may comprise a mixture of a plurality of peptide sequences consisting of any one or more of SEQ ID NO(s): 1-41 or a mixture of a plurality of peptide sequences consisting of any one or more of SEQ ID NO(s): 2, 4, 7, 9, 15, 18, 19, 20, 26, 28, 31, 35, or 39, SEQ ID NO(s): 2, 4, 7, 9, 15, 18, 19, 20, 26, 28, 31, and 35-40, or SEQ ID NO(s): 19, 34, and 35. A vaccine may comprise a mixture of a plurality of peptides consisting of each of SEQ ID NO(s): 2, 4, 7, 9, 15, 18, 19, 20, 26, 28, 31, and 35-40 or a mixture of plurality of peptides consisting of each of SEQ ID NO(s): 1-41 or a mixture of plurality of peptides consisting of each of SEQ ID NO(s): 19, 34, and 35, or any combination of the listed sequences, including a combination of two, three, four, five, six, seven, eight, nine, ten, eleven, twelve, thirteen, fourteen, or up to 20 or more sequences.

A vaccine may comprise an approximately equal molar mixture of isolated or synthesized peptides of SEQ ID NO(s): 2, 4, 7, 9, 15, 18, 19, 20, 26, 28, 31, and 35-40, an approximately equal molar mixture of isolated or synthesized peptides of SEQ ID NO(s): 2, 4, 7, 9, 15, 18, 19, 20, 26, 28, 31, 35, and 39, or an approximately equal molar mixture of the isolated or synthesized peptides of SEQ ID NO(s): 1-41. A vaccine may comprise approximately equal weight of any isolated or synthesized peptides. A vaccine may comprise an approximately equal molar mixture of any two or more of the isolated or synthesized peptides of SEQ ID NO(s): 1-41.

A pharmaceutical composition or vaccine may comprise a pharmaceutically-acceptable carrier and/or adjuvant and/or excipient. An adjuvant may be a UTOPE. A TUOPE adjuvant may be covalently attached to an isolated or synthesized peptide at the C-terminus, the N-terminus, or both termini. A UTOPE is a peptide sequence of 6 to 10 residues comprising one histidine residue with all other residues being lysine residues.

A non-limiting acceptable carrier, adjuvant, or excipient may include sterile water, oil and water emulsion, keyhole limpet hemocyanin. A non-limiting carrier, excipient, or adjuvant may include a sterile diluent such as water (for dermal, nasal, or ocular application, spraying, or injection), saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. pH can be adjusted with acids or bases, such as hydrochloric acid or sodium hydroxide. Preparations may be enclosed in ampoules, disposable syringes or multiple dose vials made of glass or plastic.

Pharmaceutical compositions suitable for use typically include sterile aqueous solutions (water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In general, a relevant carrier can be a solvent or dispersion medium containing, for example, water, ethanol, polyol (for example, glycerol, propylene glycol, and liquid polyetheylene glycol, and the like), and suitable mixtures thereof.

### Binding Molecules

Binding molecules are provided as an aspect of the invention to target Replikin structures in Filoviruses, including Ebolaviruses and Marburg viruses. Replikin peptides may be used to generate antibodies, antibody fragments, or to generate or identify other binding agents, which may be used, for example for diagnostic purposes or to provide passive immunity in an individual. See, e.g., US 2007/0026009 and US 2009/0017052 (each incorporated herein by reference in their entirety).

Various procedures known in the art may be used for the production of antibodies to Replikin sequences or to proteins, protein fragments, polypeptides, or peptides comprising Replikin sequences. Such antibodies include, but are not limited to, polyclonal, monoclonal, chimeric, humanized, single chain, Fab fragments and fragments produced by a Fab expression library. Antibodies that are linked to a cytotoxic agent may also be generated. Antibodies may also be administered in combination with an antiviral agent. Furthermore, combinations of antibodies to different Replikins may be administered as an antibody cocktail.

For the production of antibodies, various host animals or plants may be immunized by injection with a Replikin peptide or a combination of Replikin peptides, including, but not limited to, rabbits, mice, rats, and larger mammals. Monoclonal antibodies to Replikins may be prepared using any technique that provides for the production of antibody molecules. These include but are not limited to the hybridoma technique originally described by Kohler and Milstein, (Nature, 1975, 256:495-497), the human B-cell hybridoma technique (Kosbor et al., 1983, Immunology Today, 4:72), and the EBV hybridoma technique (Cole et al., Monoclonal Antibodies and Cancer Therapy, Alan R. Liss, Inc., pp. 77-96). In addition, techniques developed for the production of chimeric antibodies (Morrison et al., 1984, Proc. Nat. Acad. Sci USA, 81:6851-6855) or other techniques may be used. Alternatively, techniques described for the production of single chain antibodies (U.S. Pat. No. 4,946,778) can be adapted to produce Replikin-specific single chain antibodies. Antibody fragments that contain binding sites for a Replikin may be generated by known techniques. For example, such fragments include but are not limited to F(ab′)2 fragments which can be produced by pepsin digestion of the antibody molecules and the Fab fragments that can be generated by reducing the disulfide bridges of the F(ab′)2 fragments. Alternatively, Fab expression libraries can be generated (Huse et al., 1989, Science, 246:1275-1281) to allow rapid and easy identification of monoclonal Fab fragments with the desired specificity.

A non-limiting aspect of the present invention provides an isolated, chemically-synthesized, or recombinantly-generated binding molecule that specifically binds a Replikin sequence of a Filovirus, including an Ebolavirus or Marburg virus, and/or that specifically binds a homologue of a Replikin sequence of a Filovirus, including an Ebolavirus or Marburg virus, wherein the homologue is at least 50%, 60%, 70%, 80%, 90%, 95%, or more homologous with a Replikin sequence of a Filovirus. In a non-limiting embodiment, the Replikin sequence is not SEQ ID NO: 14, is not a Replikin sequence from a polymerase protein, and/or is not a Replikin sequence from an RNA polymerase protein.

A binding molecule may specifically bind at least one sequence of SEQ ID NO(s): 1-41 or a homologue of at least one sequence of SEQ ID NO(s): 1-41, wherein said homologue is at least 70%, 80%, 90%, 95%, or more homologous. A binding molecule may specifically bind at least one sequence of SEQ ID NO(s): 2, 4, 7, 9, 15, 18, 19, 20, 26, 28, 31, or 35-41.

A binding molecule may be any molecule that preferentially binds a Replikin sequence or homologue thereof. A binding molecule may be ligand. A binding molecule may be an antibody or antibody fragment. An antibody fragment may be an F(ab′)2 fragment or Fab fragment or any fragment of an antibody capable of specifically binding a Replikin sequence or homologue thereof. An antibody may be a monoclonal antibody. An antibody may be a humanized antibody. An antibody may be an optimized antibody. In a non-limiting embodiment, the antibody may be an fc-optimized antibody. In a non-limiting embodiment, the antibody is selected using phage display. In a non-limiting embodiment, the antibody is produced from a microorganism such as E. coli.

In a non-limiting embodiment, an antibody, antibody fragment, or binding molecule binds to at least a portion of an amino acid sequence of at least one protein, protein fragment, polypeptide, or peptide comprising a peptide sequence that is 30%, 40%, 50%, 60%, 70%, 80%, 90%, or 95% or more homologous with at least one Replikin peptide sequence identified in a Filovirus, including but not limited to Ebolavirus or Marburg virus. An antibody, antibody fragment, or binding agent may be isolated or synthesized. An antibody or antibody fragment may be a monoclonal antibody or monoclonal antibody processed to create an antibody fragment. An antibody or antibody fragment may be made from any biological or chemical method.

### Filovirus Protein Fragments and Peptides

Protein fragments and peptide comprising a Replikin sequence are understood in the art to present the Replikin sequence to an immune system for processing. As a result, an isolated or synthesized protein, protein fragment, or peptide comprising at least one peptide sequence that is at least 50%, 60%, 70%, 80%, 90%, or 95% or more homologous with at least one Replikin peptide sequence identified in a Filovirus, including an Ebolavirus or a Marburg virus, provides a target for control of replication, virulence, and lethality of a Filovirus. As the size of a protein, protein fragment, or peptide decreases with respect to the size of a Replikin sequence comprised within the protein, protein fragment, or peptide, the more the functional structure of the Replikin sequence is exposed to the immune system for stimulating an immune response against the Replikin sequence target and the more the Replikin sequence interacts with replication mechanism to block replication. A protein fragment or peptide may consist of a Replikin sequence, may consist essentially of a Replikin sequence, may consist of up to 50 amino acid residues comprising a Replikin sequence, may consist of up to 60, 70, 75, 80, 90, or 100 or more amino acid residues comprising a Replikin sequence. A peptide of 50 residues is understood to present a Replikin sequence comprised within the peptide to the immune system without interference.

Proteins, protein fragments, and peptides comprising a Replikin sequence may be identified in any Filovirus. Filoviruses include Ebolaviruses, Marburg viruses, and Ebola-like viruses. The Ebolavirus viruses include, but are not limited to, Ebola Reston virus, Ebola Sudan virus, Ebola Guinea virus, Ebola Sierra (Zaire) virus, Bundibugyo virus, Taï Forest, and Ebola virus. A Replikin peptide sequence may be identified in any Ebolavirus or Marburg virus (or other Filovirus) genome including but not limited to any expressed protein sequence. Expressed protein sequences may be identified in polymerase cofactor (VP35 and VP 40); RNA polymerase; nucleoprotein (NP); glycoprotein (GP), transcription activator (VP30), and VP24, or any other expressed protein sequence. In a non-limiting embodiment, the Replikin peptide sequence may exclude Replikin peptide sequences from RNA polymerase protein. In a non-limiting embodiment, the Replikin peptide sequence may include any Replikin peptide sequence excluding KKHATVLK (SEQ ID NO: 14).

Functional fragments of a Replikin sequence from a Filovirus such as an Ebolavirus or Marburg virus are likewise useful for targeting rapid replication, virulence, and lethality.

An isolated or synthesized protein fragment or peptide comprising a Replikin sequence or a homologue of a Replikin sequence identified in a Filovirus (where said homologue is at least 50%, 60%, 70%, 80%, 90%, 95%, 97% or greater homologous with the Replikin sequence) may likewise consist of up to 125, 150, 175, or 200 amino acid residues in total; or up to 250, 300, 350, or 400 amino acid residues in total.

An isolated or synthesized protein fragment or peptide may comprise at least one Replikin peptide sequence of any of SEQ ID NO(s): 1-13 and 15-41 or at least one homologue of SEQ ID NO(s): 1-13 and 15-41, may consists essentially of at least one Replikin peptide sequence of SEQ ID ON(s): 1-13 or 15-41 or at least one homologue of SEQ ID NO(s): 1-13 and 15-41, and may consist of at least one Replikin peptide sequence of SEQ ID NO(s): 1-13 and 15-41 or at least one homologue of SEQ ID NO(s): 1-13 and 15-41. A functional fragment of at least one Replikin peptide sequence of SEQ ID NO(s): 1-13 and 15-41 may likewise be used in a place of a Replikin sequence for preventive, therapeutic, and diagnostic purposes.

An isolated or synthesized peptide may consist of 7 to about 50 amino acids comprising at least one peptide A, wherein said peptide A is at least 30%, 40%, 50%, 60%, 70%, 80%, 90%, 95%, or 100%, homologous with at least one Replikin peptide sequence identified in a Filovirus, such as an Ebolavirus or Marburg virus. The at least one Replikin peptide sequence or homologue thereof identified in a Filovirus may be at least one peptide sequence of SEQ ID NO(s): 1-13 and 15-41 or a homologues of SEQ ID NO(s): 1-13 and 15-41.

As demonstrated in Tables 12 and 13 Replikin sequences are conserved and shared among isolates isolated from different Ebolavirus viruses and Marburg viruses as well as Filoviruses generally. The Replikin sequences may be identical within different isolates or may be homologous among different isolates. Often, functional homologues are different from a Replikin sequence at a single amino acid residue. Replikin sequences may be conserved over one, two, three, or more years.

A biosynthetic composition is provided comprising a protein, protein fragment, polypeptide, or peptide comprising a Replikin sequence identified in a Filovirus or a homologue of a Replikin sequence identified in a Filovirus. An isolated protein, protein fragment, polypeptide, or peptide may be chemically synthesized by solid phase methods.

### Methods of Making a Pharmaceutical Composition

Pharmaceutical compositions and vaccines against Filoviruses may be manufactured following identification of one or more Replikin sequences in a Filovirus. To manufacture a pharmaceutical composition or vaccine, one or more Replikin sequences are identified. A protein, protein fragment, or peptide comprising the Replikin sequence may be synthesized or isolated. Synthesis via solid state provides synthetic peptides without the contamination present in traditional vaccines. Proteins, protein fragments, or peptides are then comprised in a pharmaceutically-acceptable carrier. Administration of the composition or vaccine to a subject is available for stimulating an immune response and/or providing a blocking effect within the subject against a Filovirus. A method of making a pharmaceutical composition or vaccine may comprise: selecting at least one isolated or synthesized protein, protein fragment, polypeptide, or peptide comprising at least one peptide sequence that is at least 30%, 40%, 50%, 60%, 70%, 80%, 90% or 95%, or 100% homologous with at least one Replikin peptide sequence identified in a Filovirus as a component of a pharmaceutical composition or vaccine; and making said pharmaceutical composition or vaccine. The method of making may comprise selecting at least one isolated or synthesized peptide of SEQ ID NO(s): 1-41, as at least one component, and making said composition or vaccine with the at least one component and a pharmaceutically-acceptable carrier, adjuvant, or excipient. A pharmaceutically-acceptable carrier may include, but is not limited to, sterile water or PBS. An isolated or synthesized peptide may comprise any one of SEQ ID NO(s): 1-13 and 15-41 and may be up to 50, 60, 70, 80, 90, 100, 150, 200, or 250 residues in length.

A method of making a pharmaceutical composition or vaccine may comprise selecting at least two, three, four, five, six, seven, eight, nine, ten, eleven, twelve, thirteen, fourteen, fifteen, sixteen, seventeen, or twenty or more isolated or synthesized Replikin peptide sequences identified in a Filovirus and/or isolated or synthesized functional fragments of Replikin peptide sequences identified in a Filovirus. Isolated or synthesized Replikin peptide sequences or functional fragments of Replikin peptide sequences identified in a Filovirus may comprise at least one peptide sequence of SEQ ID NO(s): 1-13 and 15-41, at least one functional fragment of at least one peptide sequence of SEQ ID NO(s): 1-13 and 15-41, or at least one functional fragment of at least one Replikin peptide sequence identified in a Filovirus and may consist of said sequences an may comprise said sequences and be up to 50, 60, 70, 80, 90, 100, 150, 200, 250 or more residues in length. At least one isolated or synthesized protein, protein fragment, polypeptide, or peptide may have the same amino acid sequence as at least one protein, protein fragment, polypeptide or peptide identified in a relatively lethal strain of a Filovirus up to seven days, one month, six months, one year, two years, or three years prior to making said vaccine.

### Methods of Preventing or Treating Filovirus Infection, Including Ebolavirus and Marburg Virus Infections

Also provided is a method for preventing or treating Filovirus infection comprising administering at least one isolated or synthesized protein, protein fragment, polypeptide, or peptide comprising at least one peptide sequence to subject, including, for example, an animal or human, where the peptide sequence is at least 30%, 40%, 50%, 60%, 70%, 80%, 90% or 95%, or 100%, homologous with at least one Replikin peptide identified in a Filovirus. In one non-limiting embodiment, the Replikin peptide sequence is not SEQ ID NO: 14. In one non-limiting embodiment, the Replikin peptide sequence is not from a polymerase protein or an RNA polymerase protein. In a further non-limiting embodiment, the Replikin peptide sequence is at least one peptide sequence of SEQ ID NO(s): 1-13 and 15-41. In a non-limiting embodiment, the at least one isolated or synthesized protein fragment, polypeptide, or peptide consists of at least one peptide sequence that is at least 30%, 40%, 50%, 60%, 70%, 80%, 90%, or 95% or more homologous with at least one of the peptide sequences of SEQ ID NO(s): 1-13 and 15-41. In another non-limiting embodiment, the at least one isolated or synthesized peptide of SEQ ID NO(s): 1-13 and 15-41 is administered to a human, ape, chimpanzee, other primate, or other animal. In a further non-limiting embodiment the at least one Replikin peptide sequence is at least one peptide sequence of SEQ ID NO(s): 1-13 and 15-41. In a further non-limiting embodiment, a protein fragment comprising a Replikin peptide sequence, homologue thereof, or fragment thereof may be up to 50 residues in length; up to 60, 70, 80, 90, or 100 residues in length; or up to 150 or 200 or more residues in length.

### Methods of Predicting Outbreaks of Filovirus

A non-limiting aspect of the present invention provides a method of predicting expansion or retraction of a population of Filovirus comprising, respectively, identifying an increase in the percentage of isolates of Filovirus having a Replikin concentration (number of Replikin sequences per 100 amino acid residues) in the genome greater than 4.0 between two time periods or identifying a decrease in the percentage of isolates of Filovirus having a Replikin concentration (number of Replikin sequences per 100 amino acid residues) greater than 4.0 between two time periods. In a non-limiting embodiment, the more than two time periods are compared and the percentage of isolates in the more than two time periods shows a pattern of increase or decrease.

A non-limiting embodiment provides a method of differentiating between relatively more lethal or more virulent and relatively less lethal or less virulent forms of Filovirus. A first non-limiting embodiment provides a method of identifying and/or diagnosing a relatively more lethal or more virulent form of Filovirus comprising determining the Replikin concentration of at least one portion of at least one protein of at least one isolate of Filovirus or at least one portion of at least one gene that expresses at least one protein of the at least one isolate of Filovirus and comparing the Replikin concentration of the at least one isolate of Filovirus to a comparable Replikin concentration in at least one other isolate of Filovirus. In a further non-limiting embodiment, the at least one portion of at least one protein comprises the entirety of at least one protein expressed in a Filovirus and the comparable Replikin concentration is the Replikin concentration of the entirety of the same protein expressed in a Filovirus from the at least one other isolate of the same Filovirus. In a non-limiting embodiment, the Replikin concentration of the at least one isolate of Filovirus is a mean of Replikin concentrations determined in a plurality of isolates of the same Filovirus. In a further non-limiting embodiment, the Replikin concentration of the at least one other isolate of Filovirus is a mean of Replikin concentration determined in a plurality of other isolates of the same Filovirus. In a further non-limiting embodiment, the plurality of isolates of Filovirus is a collection of isolates isolated in a given year and the plurality of other isolates of Filovirus is a collection of isolates of the same Filovirus isolated in a different year. In a further non-limiting embodiment, the Replikin concentration of the more lethal isolate of Filovirus is 3.0 or greater, 4.0 or greater, 5.0 or greater, 6.0 or greater, 8.0 or greater, 10.0 or greater, or 12.0 or greater per 100 amino acid residues. In a further non-limiting embodiment, the Replikin concentration of the more lethal isolate of Filovirus is 4.0 or greater per 100 amino acid residues. In a further non-limiting embodiment, a vaccine is manufactured following the differentiation between relatively more lethal and relatively less lethal forms of Filovirus. In a further non-limiting embodiment, a vaccine is manufactured following prediction of an outbreak of Filovirus following identification of a more lethal or more virulent form of a Filovirus. In a further non-limiting embodiment, the vaccine comprises at least one structure of the isolate of Filovirus differentiated as relatively more lethal or virulent. In a further non-limiting embodiment, the vaccine comprises at least one Replikin peptide sequence identified in the isolate of Filovirus differentiated as relatively more lethal or virulent.

In a further non-limiting embodiment, the Replikin concentration of the at least one isolate of Filovirus is greater than the Replikin concentration of the at least one other isolate of Filovirus. In a further non-limiting embodiment the Replikin concentration is a mean Replikin concentration of a plurality of isolates with standard deviation from the mean and the standard deviation from the mean is greater than the standard deviation from the mean Replikin concentration of a plurality of other isolates.

Another non-limiting embodiment provides a method of determining an increased probability of an outbreak of Filovirus within about one year following an increase in Replikin concentration in an isolate of Filovirus comprising identifying an increase in the concentration of Replikin sequences in at least one first isolate of a Filovirus as compared to at least one other isolate of the same kind of Filovirus wherein said at least one first isolate is isolated at a later time period than said one other isolate and wherein said increase in the concentration of Replikin sequences signifies the increased probability of the outbreak of the Filovirus within about one year following the increase in the concentration of Replikin sequences.

In a non-limiting embodiment, a method of prediction comprises: (1) obtaining a plurality of isolates of a Filovirus wherein at least one of said isolates is isolated about six months to about 3 years later than at least one other of said isolates; (2) analyzing the amino acid sequence of at least one protein or protein fragment in each isolate of the plurality of isolates for the presence and concentration of Replikin sequences; (3) comparing the concentrations of Replikin sequences in the at least one protein or protein fragment in each isolate of the plurality of isolates one to another; (4) identifying an increase in the concentration of Replikin sequences in said plurality of isolates over at least one time period of about six months or greater; and (5) predicting an outbreak of the Filovirus within about one month to about three years following said identified increase in the concentration of Replikin sequences. In another embodiment of the invention, the outbreak of Filovirus is predicted within about six months. In a further embodiment of the invention, the outbreak of Ebola virus is predicted within about one year to about three years. In a further non-limiting embodiment, the method of prediction further comprises processing at least one step of the method on a computer.

In a further non-limiting embodiment, the protein or protein fragment is from VP35, polymerase, or glycoprotein protein of Ebolavirus.

In a further non-limiting embodiment, the method of prediction further comprises comparison of the standard deviation from the mean of Replikin concentrations of isolates of Filovirus from a given time period, such as a given month, a given year, or any other given time period. In a further non-limiting embodiment, the Replikin concentration is a mean Replikin concentration of a plurality of isolates with standard deviation from the mean and the standard deviation from the mean is greater than the standard deviation from the mean Replikin concentration of a plurality of other isolates.

A further non-limiting embodiment provides a computer readable medium having stored thereon or signal containing instructions which, when executed, cause a processor to perform a method of predicting an expansion of a strain of Filovirus or an increase in virulence, morbidity, lethality, and/or mortality of Filovirus. In a further embodiment, the processor reports a prediction to a display, user, researcher, or other machine or person. In a further embodiment, the processor identifies to a display, user, researcher, or other machine or person, a portion of a pathogen predicted to be an expanding Filovirus or predicted to increase in virulence, morbidity, lethality, and/or mortality, wherein said portion may be employed as a therapeutic or diagnostic compound. Said portion may be a Replikin peptide or plurality of Replikin peptides or any other structure or portion of said genome of said pathogen including a Replikin Peak Gene. In a non-limiting embodiment, said portion is synthesized and prepared in a pharmaceutical composition.

Another non-limiting embodiment provides a computer system, including a processor coupled to a network and a memory coupled to the processor, the memory containing a plurality of instructions to perform a method of predicting the relative virulence of at least one first group of Filoviruses as compared to at least one second group of Filoviruses.

Another non-limiting embodiment provides a machine-readable storage medium having stored thereon executable instructions that, when executed by a processor, cause the processor to provide sufficient data to a user, a display, or a printout such that said user or a user of said display or said printout may predict the virulence or lethality of a Filovirus based on regression analysis. Another non-limiting embodiment provides a computer system, comprising: a processor coupled to a network; a memory coupled to the processor, the memory containing a plurality of instructions to perform a method of predicting the virulence or lethality of an Ebola virus based on regression analysis.

### Nucleic Acid Sequences

A non-limiting aspect of the present invention provides a nucleic acid sequence that is antisense to a nucleic acid that encodes for any Replikin peptide present in or identified in a Filovirus isolate. This may include one of SEQ ID NO(s): 1-41 or a small interfering nucleic acid sequence that interferes with a nucleic acid sequence that is 30%, 40%, 50%, 60%, 70%, 80%, 90%, or 95% or more homologous with a nucleic acid that encodes any Replikin peptide of a Filovirus including, for example, any one of SEQ ID NO(s): 1-41 or is 30%, 40%, 50%, 60%, 70%, 80%, 90%, 95% or more homologous with a nucleic acid that is antisense to a nucleic acid that encodes for any one of SEQ ID NO(s): 1-41. A nucleic acid sequence may be 21 to 150 nucleotides in length. A nucleic acid sequence may be up to 5, 10, 15, 20, 25, 30, 35, 40, 45, or 50 nucleotides in length.

### Example 1

### Increase in the Ebola Gene Replikin Concentration in 2012-2013 Predicted the Ebola Outbreak of 2014 and Subsequent Early End to Outbreak

The Replikins Global Surveillance System™ found the highest ever Ebola Gene Replikin concentration in 2012 and 2013 and predicted the worst ever global Ebola outbreak of 2014. The System likewise identified a sudden drop in Ebola Gene Replikin concentrations during 2014 and, contrary to the expectation of the public health community, predicted the outbreak's early end beginning in the last quarter of 2014.

Knowledge of the Ebola Replikin concentration increase in 2012 and 2013 would have provided one to two years to prepare and to test public health responses and vaccine candidates. The post outbreak slow reaction time was too costly in lives, illness, anxiety, diversion, and commercial disruption; and was no longer necessary in view of Replikins technology. The Replikins Global Surveillance System™ provides early warning and permits time for better-targeted public health and vaccine responses.

Specific gene sequences in the infectious organism have been found, named Replikins because of their association with rapid replication and population expansion of pathogens. The increasing gene Replikin concentration has been found to predict, one to two years in advance, outbreaks and the course and termination of virus and bacterial pathogenic activity (1) and to provide time to respond in advance of the outbreak.

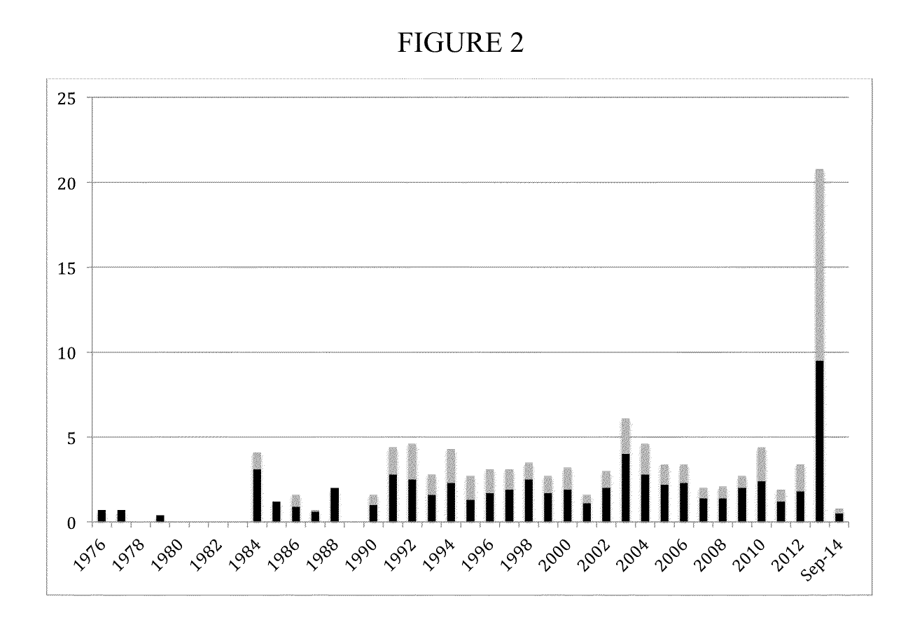

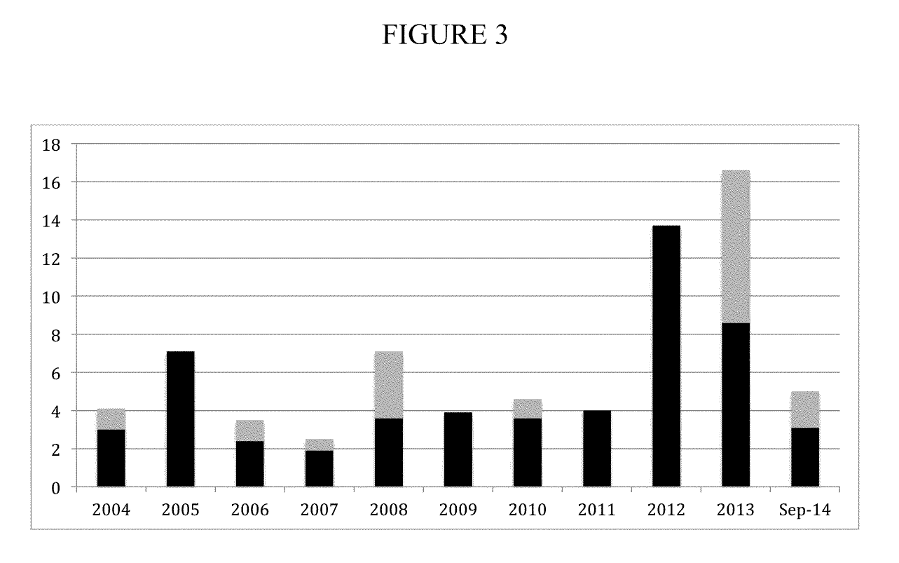

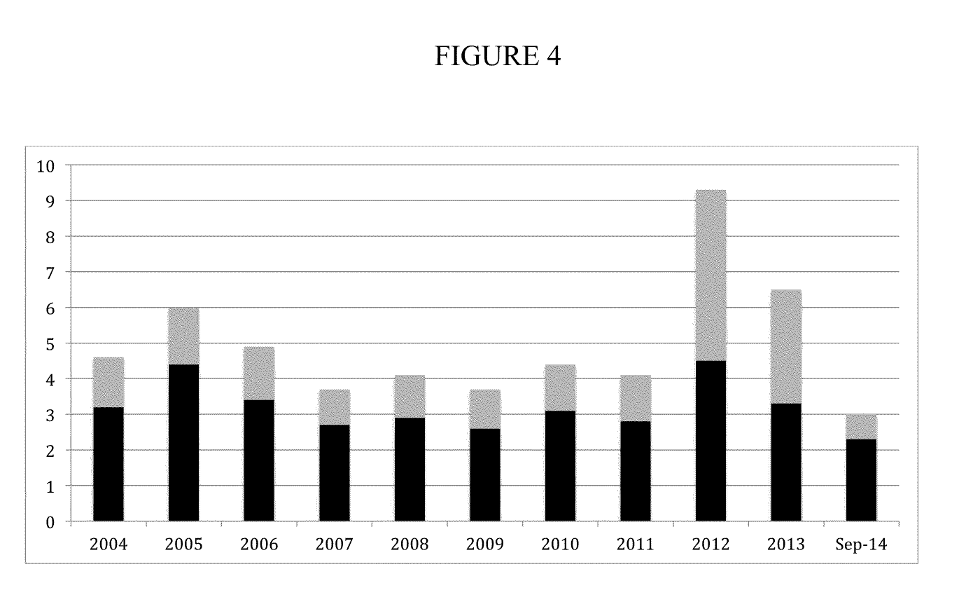

Gene changes in all Ebola sequences in the NCBI PubMed database were examined retrospectively back to 1976 when Ebola was first described, and prospectively, after the outbreak was reported in the first months of 2014. Six independent probes were performed (FIGS. 1-5 and 8) into all Ebola strains together (N=10,197), and in separate strains, and in several individual Ebola proteins (glycoprotein FIG. 2, VP35 FIG. 3, polymerase FIG. 4). Since each of the six Replikins software analyses, each performed with the same algorithm used in all such analyses over the past decade, were completely automated and independent of each other, it is notable that the same result of the rise to a 2012-2013 peak at p less than 0.001 was obtained in each probe, rising to the highest levels ever for the Ebola virus, and correlated with the worst Ebola outbreak ever in 2014. For two Ebola proteins (VP35, see FIG. 3, and polymerase, see FIG. 4) the warning came two years in advance, in 2012, and if known then, could have resulted in preparatory action.

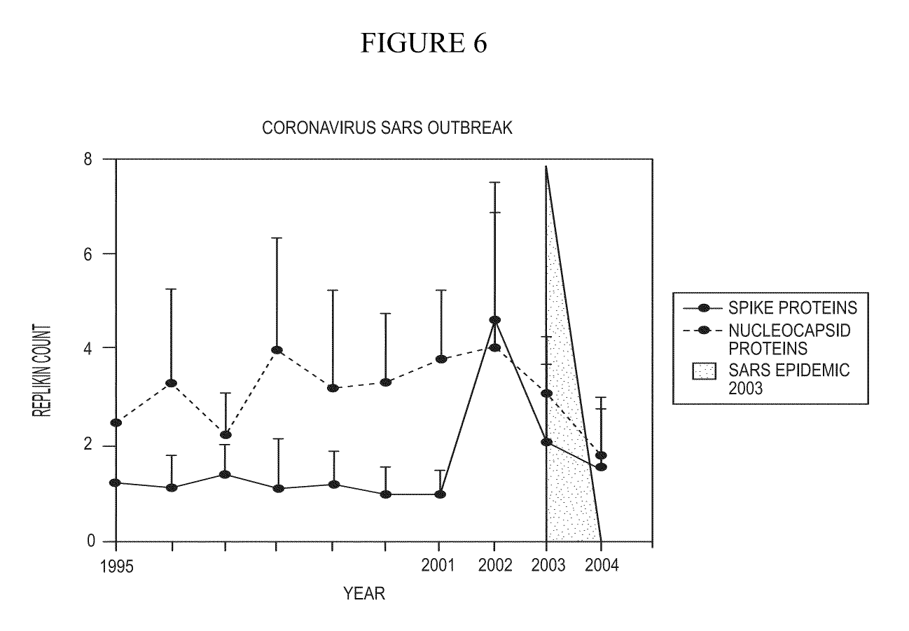

Prospectively, instead of the elevated Replikin concentration persisting, as it did in H1N1(1), the elevated gene Replikin concentration of Ebola precipitously dropped during 2014 to its pre-outbreak levels (persisting at a Replikin concentration of 0.5+/−0.2 through Apr. 5, 2015), exactly as it did in the SARS outbreak of 2003 (FIG. 6). As observed in October 2014, this drop predicted an early end to the outbreak so long as the required public health containment measures were implemented against the viruses already in human hosts to prevent their spread. FIG. 2 illustrates a marked rise observed in Replikin concentration in Ebola glycoprotein in 2012. FIG. 4 illustrates a marked rise in Replikin concentration in Ebola polymerase in 2013.

Both the rise and the fall in Replikin concentrations in Ebolaviruses are the same as found in the SARS outbreak of 2002-2003, which was over in less than one year. However, in SARS 2003, excellent intensive public health containment efforts followed the drop in the SARS gene Replikins. In Ebola, the public health containment was not very effective but the drop in virulence of the virus, as demonstrated through Replikin concentration observations, provided nevertheless-insufficient public health measures with sufficient relief to succeed in ending the outbreak.

In September 2014, the Centers for Disease Control and Prevention estimated that by mid-January 2015 the number of Ebola cases could have been as many as 1.4 million. The number of Ebola cases reported Jan. 28, 2015 was magnitudes less, namely, 22,057. These data demonstrate the huge difference between expectations in public health and the predictive capacity of Replikins technology, which predicted in October 2014 that the outbreak would soon diminish.

As calculated from the rates of appearance of new Ebola cases in October 2014, the Ebola outbreak was estimated by public health officials to last an additional eighteen months or longer. In contrast to this calculation, the observed drop in gene Replikin concentration during 2014 predicted termination of the outbreak and predicted that termination was only a few months away. This is comparable to how the gene Replikin concentration dropped in SARS in 2003. The sharp drop in the Ebola gene Replikin concentration observed in 2014 indicated that the gene reservoir for rapid replication had declined in both animal and human hosts. These data should have encouraged the containment efforts. Strengthened quarantine and travel restrictions were recommended to isolate those Ebola viruses that had already entered human hosts in order to hasten the end of the Ebola outbreak.

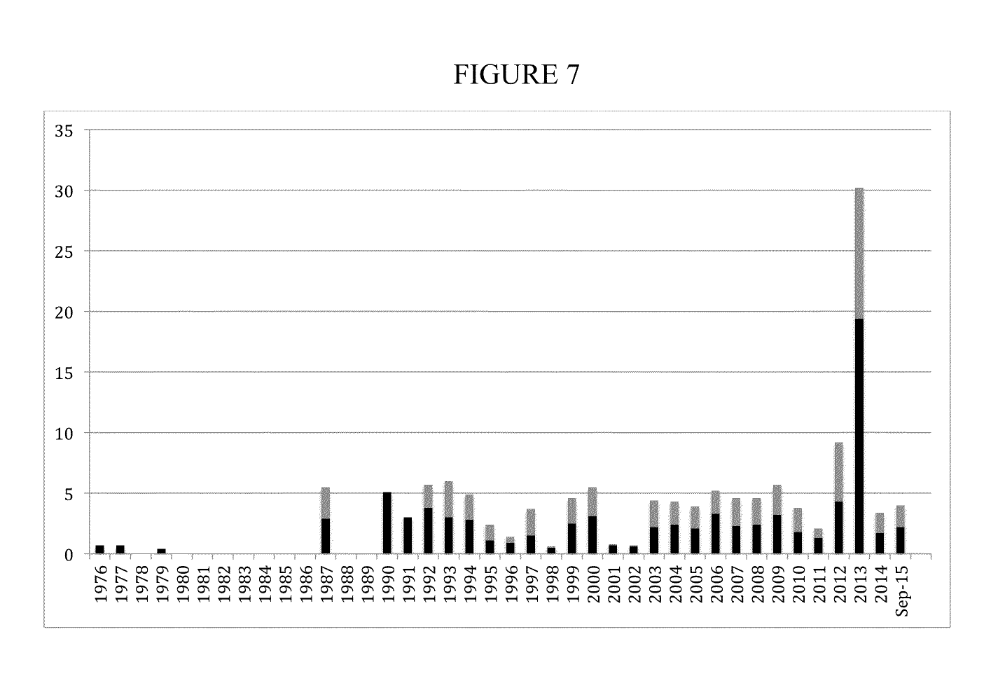

It is noteworthy that a three-fold increase in Replikin concentration was identified in May 2015 with a p value of less than 0.0001. See, e.g., FIG. 7. As such, Replikin concentration in Ebolaviruses is building again in 2015.

The Replikins Global Surveillance System™, organized by Replikins, Ltd. has been designed to provide early response based on understanding of quantitative changes in the genome prior to outbreaks and epidemics in animal and human hosts. Outbreaks have followed when the Replikin concentration increase reaches a statistical level of significance of p<0.001. Gene Replikin concentration increase successfully predicted in 2008, one year in advance, the H1N1 influenza pandemic of 2009. The H5N1 outbreaks in Indonesia and Cambodia, 2007 to 2012 were among 13 others also anticipated and localized geographically by the gene Replikin concentration increase (1).

In response to the early warning, public health measures and vaccines can be tested in advance of the outbreak. Emerging diseases thus are being detected real-time, early, before major outbreaks, to the potential benefit of individual countries in which they originate and with the ultimate goal of pandemic prevention. The earliest possible submission of specimens from initial cases for sequence Replikins analysis will increase the time available for constructing an accurate, safe, and effective response.

### REFERENCES

1. Bogoch S and Bogoch E S. Prediction of specific virus outbreaks made from the increased concentration of a new class of virus genomic peptides, Replikins. Nature Precedings doi: 10.1038/npre2011.6279.1
2. Jackwood M W et al. Efficacy of a Replikin Peptide Vaccine Against Low Pathogenicity Avian Influenza H5 Virus. Avian Diseases 53(4): 613-617, 2009.

FIGS. 1-5 and 8 and related analyses were generated from the data provided in the following tables (Tables 1-11).

TABLE 1
Mean Replikin Concentration per Year of Ebola Virus Sequences
from NCBI Database as of 9 Sep. 2014 (see FIG. 1)

| | Mean Replikin | Standard | Number of |
|---|---|---|---|
| Year | concentration | Deviation | Sequences |
| 1976 | 1.4 | 1 | 2 |
| 1977 | 1.4 | 1 | 2 |
| 1978 | | | |
| 1979 | 0.9 | 0.7 | 2 |
| 1980 | | | |
| 1981 | | | |
| 1982 | | | |
| 1983 | 0.7 | 0 | 1 |
| 1984 | 3.1 | 1 | 2 |
| 1985 | 1.2 | 0.7 | 3 |
| 1986 | 0.9 | 0.7 | 8 |
| 1987 | 3.7 | 3.5 | 13 |
| 1988 | 1 | 0.7 | 4 |
| 1989 | | | |
| 1990 | 1.5 | 1.6 | 8 |
| 1991 | 2.8 | 1.4 | 8 |
| 1992 | 2.8 | 2.1 | 20 |
| 1993 | 2.2 | 1.5 | 31 |
| 1994 | 2.5 | 1.9 | 108 |
| 1995 | 1.8 | 1.7 | 30 |
| 1996 | 2.1 | 1.6 | 47 |
| 1997 | 1.9 | 1.1 | 258 |
| 1998 | 2.4 | 1.1 | 64 |
| 1999 | 2.1 | 1.7 | 128 |
| 2000 | 2.6 | 2.1 | 159 |
| 2001 | 1.4 | 1 | 81 |
| 2002 | 2 | 1 | 640 |
| 2003 | 3.9 | 2.1 | 324 |
| 2004 | 2.8 | 1.7 | 365 |
| 2005 | 2.5 | 1.5 | 174 |
| 2006 | 2.2 | 0.9 | 582 |
| 2007 | 2 | 1.3 | 362 |
| 2008 | 2.4 | 2.6 | 193 |
| 2009 | 2.2 | 1.2 | 275 |
| 2010 | 2.6 | 1.9 | 256 |
| 2011 | 1.6 | 1.3 | 253 |
| 2012 | 2.4 | 2.8 | 140 |
| 2013 | 3.6 | 5.3 | 244 |
| Sep-14 | 2.4 | 2.3 | 1033 |
| | | Total | 5820 |
| | | Number of | |
| | | Sequences | |
| | | (N) | |

TABLE 2
Ebola Virus Sequences from NCBI Database
Mean Replikin Concentration Compared to Prior
Year [% Increase (Inc.) or % Decrease (Dec.)]

| | | Change in |
|---|---|---|
| | Change in | Standard |
| | Mean from | Deviation from |
| Year | Prior Year | Prior Year |
| 2012 | Inc. 50% | Inc. 125% |
| 2013 | Inc. 50% | Inc. 89% |
| September 2014 | Dec. 33.3% | Dec. 57% |

TABLE 3
Mean Replikin Concentration per Year of Ebola Virus Glycoprotein
Sequences from NCBI Database as of 9 Sep. 2014 (see FIG. 2)

| | Mean Replikin | Standard | Number of |
|---|---|---|---|
| Year | concentration | Deviation | Sequences |
| 1976 | 0.7 | 0 | 1 |
| 1977 | 0.7 | 0 | 1 |
| 1978 | | | |
| 1979 | 0.4 | 0 | 1 |
| 1980 | | | |
| 1981 | | | |
| 1982 | | | |
| 1983 | | | |
| 1984 | 3.1 | 1 | 2 |
| 1985 | 1.2 | 0 | 1 |
| 1986 | 0.9 | 0.7 | 8 |
| 1987 | 0.6 | 0.1 | 6 |
| 1988 | 2 | 0 | 1 |
| 1989 | | | |
| 1990 | 1 | 0.6 | 6 |
| 1991 | 2.8 | 1.6 | 7 |
| 1992 | 2.5 | 2.1 | 15 |
| 1993 | 1.6 | 1.2 | 13 |
| 1994 | 2.3 | 2 | 88 |
| 1995 | 1.3 | 1.4 | 28 |
| 1996 | 1.7 | 1.4 | 35 |
| 1997 | 1.9 | 1.2 | 212 |
| 1998 | 2.5 | 1 | 38 |
| 1999 | 1.7 | 1 | 117 |
| 2000 | 1.9 | 1.3 | 109 |
| 2001 | 1.1 | 0.5 | 63 |
| 2002 | 2 | 1 | 594 |
| 2003 | 4 | 2.1 | 300 |
| 2004 | 2.8 | 1.8 | 326 |
| 2005 | 2.2 | 1.2 | 150 |
| 2006 | 2.3 | 1.1 | 321 |
| 2007 | 1.4 | 0.6 | 183 |
| 2008 | 1.4 | 0.7 | 146 |
| 2009 | 2 | 0.7 | 254 |
| 2010 | 2.4 | 2 | 220 |
| 2011 | 1.2 | 0.7 | 186 |
| 2012 | 1.8 | 1.6 | 41 |
| 2013 | 9.5 | 11.3 | 28 |
| Sep-14 | 0.5 | 0.3 | 313 |
| | | Total | 3814 |
| | | Number of | |
| | | Sequences | |
| | | (N) | |

TABLE 4
Mean Replikin Concentration per Year of Ebola Virus VP35 Protein
Sequences from NCBI Database as of 9 Sep. 2014 (see FIG. 3)

| | Mean Replikin | Standard | Number of |
|---|---|---|---|
| Year | concentration | Deviation | Sequences |
| 1990 | 5.1 | 0 | 1 |
| 1991 | | | |
| 1992 | 4.1 | 1 | 5 |
| 1993 | 1.5 | 0 | 3 |
| 1994 | 3.4 | 1.5 | 8 |
| 1995 | 1.5 | 0 | 2 |
| 1996 | | | |
| 1997 | 0.9 | 0.5 | 4 |
| 1998 | | | |
| 1999 | 2.3 | 2 | 19 |
| 2000 | 1.5 | 0 | 1 |
| 2001 | 2.5 | 0.5 | 2 |
| 2002 | 1.8 | 0.4 | 4 |
| 2003 | 1.5 | 0 | 1 |
| 2004 | 3 | 1.1 | 4 |
| 2005 | 7.1 | 0 | 1 |
| 2006 | 2.4 | 1.1 | 4 |
| 2007 | 1.9 | 0.6 | 5 |
| 2008 | 3.6 | 3.5 | 5 |
| 2009 | 3.9 | 0 | 2 |
| 2010 | 3.6 | 1 | 21 |
| 2011 | 4 | 0 | 1 |
| 2012 | 13.7 | 0 | 5 |
| 2013 | 8.6 | 8 | 10 |
| Sep-14 | 3.1 | 1.9 | 41 |
| | | Total | 149 |
| | | Number of | |
| | | Sequences | |
| | | (N) | |

TABLE 5
VP35 Protein Ebola Virus Sequences from
NCBI Database Mean Replikin Concentration
Compared to Prior Year [% Increase (Inc.) or % Decrease (Dec.)]

| | | Change in |
|---|---|---|
| | Change in | Standard |
| | Mean from | Deviation from |
| Year | Prior Year | Prior Year |
| 2012 | Inc. 225% | 0 SD |
| 2013 | Dec. 37% | NA |
| September 2014 | Dec. 59% | Dec. 76% |

TABLE 6
Mean Replikin Concentration per Year of Ebola Virus
Polymerase Protein Sequences from
NCBI Database as of 9 Sep. 2014 (see FIG. 4)

| | Mean Replikin | Standard | Number of |
|---|---|---|---|
| Year | concentration | Deviation | Sequences |
| 1976 | 2.1 | 0 | 1 |
| 1977 | 2.1 | 0 | 1 |
| 1978 | | | |
| 1979 | | | |
| 1980 | | | |
| 1981 | | | |
| 1982 | | | |
| 1983 | | | |
| 1984 | | | |
| 1985 | | | |
| 1986 | | | |
| 1987 | | | |
| 1988 | | | |
| 1989 | | | |
| 1990 | 3.3 | 0 | 1 |
| 1991 | | | |
| 1992 | 4.5 | 0 | 1 |
| 1993 | 3.5 | 2.9 | 2 |
| 1994 | 3 | 1 | 12 |
| 1995 | 1.5 | 0.9 | 6 |
| 1996 | 3.1 | 1 | 5 |
| 1997 | 1.9 | 0.3 | 43 |
| 1998 | | | |
| 1999 | 2.2 | 1.6 | 20 |
| 2000 | 2.5 | 0 | 1 |
| 2001 | 3.1 | 0.7 | 5 |
| 2002 | 4.1 | 3.8 | 23 |
| 2003 | 8.8 | 13.5 | 7 |
| 2004 | 3.2 | 1.4 | 13 |
| 2005 | 4.4 | 1.6 | 7 |
| 2006 | 3.4 | 1.5 | 24 |
| 2007 | 2.7 | 1 | 21 |
| 2008 | 2.9 | 1.2 | 11 |
| 2009 | 2.6 | 1.1 | 6 |
| 2010 | 3.1 | 1.3 | 32 |
| 2011 | 2.8 | 1.3 | 17 |
| 2012 | 4.5 | 4.8 | 28 |
| 2013 | 3.3 | 3.2 | 101 |
| Sep-14 | 2.3 | 0.7 | 370 |
| | | Total | 758 |
| | | Number of | |
| | | Sequences | |
| | | (N) | |

TABLE 7
Polymerase Protein Ebola Virus Sequences from
NCBI Database Mean Replikin Concentration
Compared to Prior Year [% Increase (Inc.) or % Decrease (Dec.)]

| | | Change in |
|---|---|---|
| | Change in | Standard |
| | Mean from | Deviation from |
| Year | Prior Year | Prior Year |
| 2012 | Inc. 60.7% | Inc. 269% |
| 2013 | Dec. 27% | Dec. 33.3% |
| September 2014 | Dec. 30.3% | Dec. 78.1% |

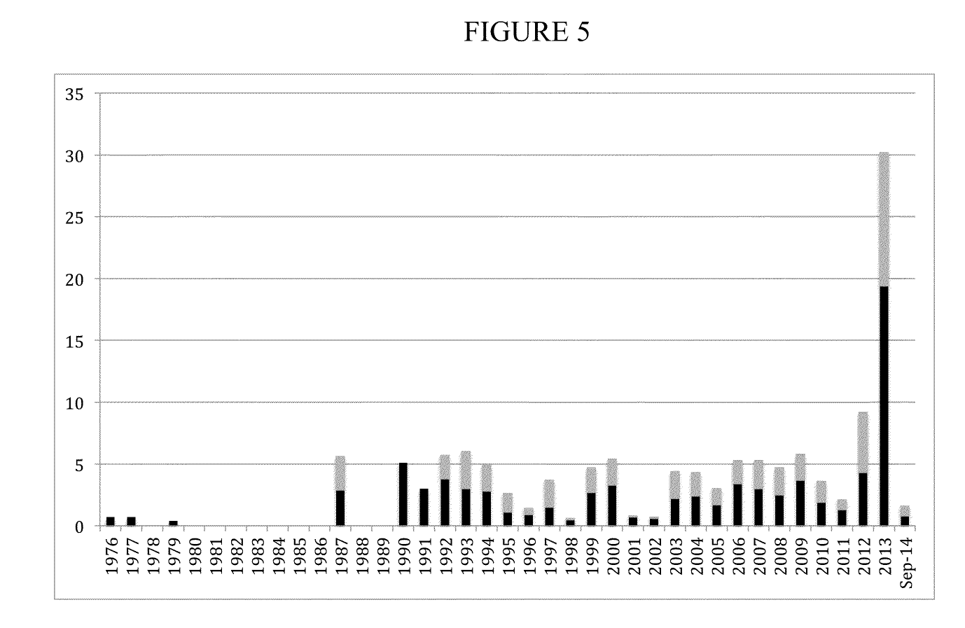

TABLE 8
Mean Replikin Concentration per Year of Ebola Marburg Virus
Sequences from NCBI Database as of 9 Sep. 2014 (see FIG. 5)

| | Mean Replikin | Standard | Number of |
|---|---|---|---|
| Year | concentration | Deviation | Sequences |
| 1976 | 0.7 | 0 | 1 |
| 1977 | 0.7 | 0 | 1 |
| 1978 | | | |
| 1979 | 0.4 | 0 | 1 |
| 1980 | | | |
| 1981 | | | |
| 1982 | | | |
| 1983 | | | |
| 1984 | | | |
| 1985 | | | |
| 1986 | | | |
| 1987 | 2.9 | 2.7 | 11 |
| 1988 | | | |
| 1989 | | | |
| 1990 | 5.1 | 0 | 1 |
| 1991 | 3 | 0 | 1 |
| 1992 | 3.8 | 1.9 | 13 |
| 1993 | 3 | 3 | 13 |
| 1994 | 2.8 | 2.1 | 22 |
| 1995 | 1.1 | 1.5 | 16 |
| 1996 | 0.9 | 0.5 | 13 |
| 1997 | 1.5 | 2.2 | 6 |
| 1998 | 0.5 | 0.1 | 2 |
| 1999 | 2.7 | 2 | 42 |
| 2000 | 3.3 | 2.1 | 109 |
| 2001 | 0.7 | 0.1 | 3 |
| 2002 | 0.6 | 0.1 | 7 |
| 2003 | 2.2 | 2.2 | 11 |
| 2004 | 2.4 | 1.9 | 21 |
| 2005 | 1.7 | 1.3 | 19 |
| 2006 | 3.4 | 1.9 | 25 |
| 2007 | 3 | 2.3 | 49 |
| 2008 | 2.5 | 2.2 | 25 |
| 2009 | 3.7 | 2.1 | 29 |
| 2010 | 1.9 | 1.7 | 10 |
| 2011 | 1.3 | 0.8 | 6 |
| 2012 | 4.3 | 4.9 | 28 |
| 2013 | 19.4 | 10.8 | 12 |
| Sep-14 | 0.8 | 0.8 | 118 |
| | | Total | 615 |
| | | Number of | |
| | | Sequences | |
| | | (N) | |

TABLE 9
Ebola Marburg Virus Sequences from NCBI
Database Mean Replikin Concentration Compared
to PriorYear [% Increase (Inc.) or % Decrease (Dec.)]

| | | Change in |
|---|---|---|
| | Change in | Standard |
| | Mean from | Deviation from |
| Year | Prior Year | Prior Year |
| 2012 | Inc. 231% | Inc. 513% |
| 2013 | Inc. 351% | Inc. 220% |
| September 2014 | Dec. 96% | Dec. 93% |

TABLE 10
Mean Replikin Concentration per Year of Ebola Marburg Virus
Sequences from NCBI Database as of September 2015 (see FIG. 7)

| | Mean Replikin | Standard | Number of | |
|---|---|---|---|---|
| Year | concentration | Deviation | Sequences | P < |
| 1976 | 0.7 | 0 | 3 | |
| 1977 | 0.7 | 0 | 1 | |
| 1978 | | | | |
| 1979 | 0.4 | 0 | 1 | |
| 1980 | | | | |
| 1981 | | | | |
| 1982 | | | | |
| 1983 | | | | |
| 1984 | | | | |
| 1985 | | | | |
| 1986 | | | | |
| 1987 | 2.9 | 2.6 | 15 | |
| 1988 | | | | |
| 1989 | | | | |
| 1990 | 5.1 | 0 | 1 | |
| 1991 | 3 | 0 | 1 | |
| 1992 | 3.8 | 1.9 | 13 | |
| 1993 | 3 | 3 | 13 | |
| 1994 | 2.8 | 2.1 | 22 | |
| 1995 | 1.1 | 1.3 | 19 | |
| 1996 | 0.9 | 0.5 | 13 | |
| 1997 | 1.5 | 2.2 | 6 | |
| 1998 | 0.5 | 0.1 | 2 | |
| 1999 | 2.5 | 2.1 | 37 | |
| 2000 | 3.1 | 2.4 | 88 | |
| 2001 | 0.7 | 0.1 | 3 | |
| 2002 | 0.6 | 0.1 | 7 | |
| 2003 | 2.2 | 2.2 | 11 | |
| 2004 | 2.4 | 1.9 | 21 | |
| 2005 | 2.1 | 1.8 | 25 | |
| 2006 | 3.3 | 1.9 | 26 | |
| 2007 | 2.3 | 2.3 | 38 | |
| 2008 | 2.4 | 2.2 | 23 | |
| 2009 | 3.2 | 2.5 | 18 | |
| 2010 | 1.8 | 2 | 7 | |
| 2011 | 1.3 | 0.8 | 6 | |
| 2012 | 4.3 | 4.9 | 28 | <0.002 |
| 2013 | 19.4 | 10.8 | 12 | <0.001 |
| 2014 | 1.7 | 1.7 | 1778 | <0.001 |
| Sep-15 | 2.2 | 1.8 | 159 | <0.001 |
| | | Total | 2397 | |
| | | Number of | | |
| | | Sequences | | |
| | | (N) | | |

As may be seen in Table 10, like Ebolavirus, after one year of low Replikin concentrations, Replikin concentration is again building in 2015 in Marburg Filovirus.

TABLE 11
Outbreaks of Ebola (Centers for Disease Control)
(virus is highlighted with country outbreaks below)

| 1976 | 1979 | 1989-1992 | 1996 | 2000-2003 | 2004 | 2007-2008 | 2014 |
|---|---|---|---|---|---|---|---|
| Zaire | Sudan | Reston | Ebola | Sudan | Ebola | Ebola | Ebola |
| Sudan | | Italy | Gabon | Uganda | Russia, | Sudan | Zaire |
| | | | | | Congo | | |
| | | USA | South | Ebola | Bundibugyo | Congo | Sierra Leone |
| | | | Africa | | | | |
| | | Philippines | Reston | Gabon | Uganda | Uganda | Multiple |
| | | | | | | | Countries |
| | | | USA | Congo | Reston | | |
| | | | Philippines | | Philippines | | |
| | | | Ebola | | | | |
| | | | Russia | | | | |

### Example 2

### Replikin Peptides Shared Among Ebolavirus Viruses

The following Replikin peptide sequences were identified as shared among Ebolavirus isolates across virus types and across different geographical regions and countries. Replikin peptide sequences conserved across geographical regions and across time are excellent sequences for targeting replication and virulence of Ebolaviruses. A vaccine may comprise any one or more of the following sequences and/or may comprise any homologue of any one or more of the following sequences. Likewise binding agents such as antibodies and other binding agents that target these sequences or their homologues are useful for controlling Ebolavirus replication.

Table 12 provides sequences identified in particular Ebolaviruses and in particular regions along with the year of isolation of the sequence in which the Replikin sequence was identified (such as 1976 or, for example, 79 or 14, which denote 1979 or 2014, respectively).

TABLE 12

| | | | Ebola |
|---|---|---|---|
| Ebola | Ebola | Ebola | Sierra |
| Reston | Sudan | Guinea | (Zaire) |
| KLFLESGAVKYLEGH | | | KLFLESGAVKYLEGH |
| (SEQ ID NO: 1) | | | (SEQ ID NO: 1) |
| 1976 | | | 14 |
| KTVLDHILQK | KTVLDHILQK | KTVLDHILQK | KTVLDHILQK |
| (SEQ ID NO: 2) | (SEQ ID NO: 2) | (SEQ ID NO: 2) | (SEQ ID NO: 2) |
| 1976 | 79 | 90 | 14 |
| HNSTLQVSDVDKLV | | | |
| CRDK | | | |
| (SEQ ID NO: 3) | | | |
| 1976 | | | |
| KAALSSLAKH | KAALGSLAKH | KAALSSLAKH | KAALSSLAKH |
| (SEQ ID NO: 4) | (SEQ ID NO: 21) | (SEQ ID NO: 4) | (SEQ ID NO: 4) |
| 1976 | 79 | 90 | 14 |
| KGFLCDSSFCKKDH | KGFLCDSNFCKKDH | KGFLCDSSFCKKDH | |
| (SEQ ID NO: 5) | (SEQ ID NO: 22) | (SEQ ID NO: 5) | |
| 1976 | 79 | 94 | |
| | | KGFLCDSNFCKKDH | |
| | | (SEQ ID NO: 22) | |
| | | 09 | |
| KKILMNFHQK | KKILMSFHQK | | KKILMNFHQK |
| (SEQ ID NO: 6) | (SEQ ID NO: 23) | | (SEQ ID NO: 6) |
| 1976 | 79 | | 14 |
| KILMNFHQK | KILMSFHQK | | KILMNFHQK |
| (SEQ ID NO: 7) | (SEQ ID NO: 24) | | (SEQ ID NO: 7) |
| 1976 | 79 | | 14 |
| KITLLTLIKTAEH | | | |
| (SEQ ID NO: 8) | | | |
| 1976 | | | |
| KILEQFHLQK | | KILEQFHLQK | |
| (SEQ ID NO: 9) | | (SEQ ID NO: 9) | |
| 1967 | | 67,87,92,04 | |
| KGGQQKNSQKGQH | | | KGGQQKNSQKGQH |
| (SEQ ID NO: 10) | | | (SEQ ID NO: 10) |
| 1976 | | | 14 |
| HHAYQGDYKLFLESG | | | |
| AVK | | | |
| (SEQ ID NO: 11) | | | |
| 1976 | | | |
| HGFRFEVKKRDGVK | | | |
| (SEQ ID NO: 12) | | | |
| 1976 | | | |
| KVKKHATVLK | | KVKKHATVLK | |
| (SEQ ID NO: 13) | | (SEQ ID NO: 13) | |
| 1994 | | 00 | |
| KKHATVLK | | KKHATVLK | |
| (SEQ ID NO: 14) | | (SEQ ID NO: 14) | |
| 1994 | | 00 | |
| KHATVLK | | KHATVLK | |
| (SEQ ID NO: 15) | | (SEQ ID NO: 15) | |
| 1994 | | 00 | |
| KGMALLHRLK | | KGMALLHRLK | KGMALLHRLK |
| (SEQ ID NO: 16) | | (SEQ ID NO: 16) | (SEQ ID NO: 16) |
| 1994 | | 04 | 93,94,14 |
| HHHHHHVDDDDKENL | | | |
| YFQSK | | | |
| (SEQ ID NO: 17) | | | |
| 2013 | | | |
| KPSQAHK | | KPSQAHK | |
| (SEQ ID NO: 18) | | (SEQ ID NO: 18) | |
| 2014 | | 14 | |
| KQTYTGIHLDK | | | KQTYTGIHLDK |
| (SEQ ID NO: 19) | | | (SEQ ID NO: 19) |
| 1967 | | | 1967 |
| KPTAPHVRNK | | KPTAPHVRNK | |
| (SEQ ID NO: 20) | | (SEQ ID NO: 20) | |
| 1967 | | 67,94,04 | |
| | | HPKLRPILLPNK | |
| | | (SEQ ID NO: 25) | |
| | | 94 | |
| | | HKGMALLHRLK | HKGMALLHRLK |
| | | (SEQ ID NO: 26) | (SEQ ID NO: 26) |
| | | 94 | 14 |
| | | KFSLLHESTLK | KFSLLHESTLK |
| | | (SEQ ID NO: 27) | (SEQ ID NO: 27) |
| | | 94 | 14 |
| | | KHIYRLK | |
| | | (SEQ ID NO: 28) | |
| | | 00 | |
| | | HIYRLKYDVTVTK | |
| | | (SEQ ID NO: 29) | |
| | | 00 | |
| | | KVKKHATVLK | |
| | | (SEQ ID NO: 13) | |
| | | 00 | |
| | | KKHATVLK | |
| | | (SEQ ID NO: 14) | |
| | | 00 | |
| | | KHATVLK | |
| | | (SEQ ID NO: 15) | |
| | | 00 | |
| | | KHNGTWQAEFK | |
| | | (SEQ ID NO: 30) | |
| | | 00 | |
| | | KFFLIVQALKH | |
| | | (SEQ ID NO: 31) | |
| | | 00 | |
| | | KQTYTGIHLDK | |
| | | (SEQ ID NO: 19) | |
| | | 67, 87, 94, and 04 | |
| | | KSQDHNSASK | |
| | | (SEQ ID NO: 32) | |
| | | 14 | |
| | | KKPGPAKFSLLH | KKPGPAKFSLLH |
| | | (SEQ ID NO: 33) | (SEQ ID NO: 33) |
| | | 14 | 14 |

As may be seen from Table 12, many isolates of Ebolavirus viruses are observed to have Replikin sequences that are exactly the same. Many viruses are additionally observed to have Replikin sequences that are exactly the same with otherwise a change in a single amino acid sequence. An example of exact conservation of Ebola viruses may be seen in SEQ ID NO: 2, which is exactly shared in Ebola Reston, Ebola Sudan, Ebola Guinea, and Ebola Sierra (Zaire) in 1976, 1979, 1990, and 2014, respectively. This sequences, therefore, is an excellent target for control of Ebola viruses.

An example of homologous conservation where a single amino acid residue is changed between isolates and regions may be seen, for example, in SEQ ID NO: 4, which is exactly conserved in Ebola Reston, Ebola Guinea, and Ebola Sierra (Zaire) and conserved but for position 5 in SEQ ID NO: 22 in Ebola Sudan. As a result, SEQ ID NO: 4 may be targeted for control of Ebola Guinea, which reflects the SEQ ID NO: 22 homologue of SEQ ID NO: 4. Likewise, SEQ ID NO: 22 may be targeted for control of Ebola Reston, Ebola Guinea, and Ebola Sierra (Zaire), which reflects the SEQ ID NO: 4 homologue of SEQ ID NO: 22.

Sharing of homologues across isolates, times, and regions provides for broad control of Ebola viruses across times, regions, and types of virus. Other examples of this sharing and homology may be seen, for example, throughout Table 12.

Table 13 provides sequences identified in particular Marburg viruses along with the year of isolation of the sequence in which the Replikin sequence was identified. These sequences and their homologues are likewise available for targeting Ebola and Marburg virus outbreaks.

TABLE 13
Marburg Virus

KIEEILSKIYH (SEQ ID NO: 34) 12, 13, 14
KVHEEMK (SEQ ID NO: 35) 13RC25.4
KQTYTGIHLDK (SEQ ID NO: 19)

### Example 3

### Synthetic Replikin Vaccine Against Ebolavirus and Marburg Virus Using Selected Conserved Peptides

A synthetic Replikin vaccine containing approximately equal-parts-by-weight of the following seventeen peptides (with specific amount of peptide synthesized) was designed and prepared for use against Ebolavirus:

| | | |
|---|---|---|
| KTVLDHILQK | (SEQ ID NO: 2) | 0.332 g |
| KAALSSLAKH | (SEQ ID NO: 4) | 0.337 g |
| KILMNFHQK | (SEQ ID NO: 7) | 0.317 g |
| KILEQFHLQK | (SEQ ID NO: 9) | 0.386 g |
| KHATVLK | (SEQ ID NO: 15) | 0.241 g |
| KPSQAHK | (SEQ ID NO: 18) | 0.258 g |
| KPTAPHVRNK | (SEQ ID NO: 20) | 0.341 g |
| KHIYRLK | (SEQ ID NO: 28) | 0.266 g |
| KFFLIVQALKH | (SEQ ID NO: 31) | 0.450 g |
| KVHEEMK | (SEQ ID NO: 35) | 0.259 g |
| KAHSDDEK | (SEQ ID NO: 36) | 0.303 g |
| HPKESRSNK | (SEQ ID NO: 37) | 0.305 g |
| KSRSAHARK | (SEQ ID NO: 38) | 0.319 g |
| KLYEAVHK | (SEQ ID NO: 39) | 0.306 g |
| KNNGTWQAEFK | (SEQ ID NO: 40) | 0.435 g |

SEQ ID NO(s): 2, 4, 7, 9, 15, 18, 19, 20, 26, 28, 31, 35, and 39 were identified as shared Replikin structures in Ebolavirus and Marburg virus. SEQ ID NO(s): 36-38 were synthesized as Replikin adjuvant homologues to Ebolavirus sequences. SEQ ID NO: 40 was synthesized as an adjuvant homologue to the Zaire Ebola sequence KHNGTWQAEFK (SEQ ID NO: 41).

The peptides were synthesized by solid-state synthesis and freeze dried. The peptides in the above-identified amounts were mixed together. A sufficient amount of mixture was dissolved in deionized water to prepare a solution having a concentration of 4 g/L.

A single rabbit was prepared for testing of the vaccine. The rabbit was bled and tested for antibodies to the mixture of peptides at various stages before, during, and after administration of the vaccine. Antibody response was determined via ELISA. The ELISA plate was prepared by adherence of the mixture of the peptides to the surface of the wells of the plate. Concentration of binding antibodies on the plate was determined via optical density.

The single rabbit was tested for antibody response prior to administration of the vaccine (Pre-bleed) on day 0.

A 25 μL aliquot of 4 g/L aqueous peptide mixture was administered to the rabbit (a 100 μg dose) intranasally on day 1. A 25 μL aliquot of 4 g/L aqueous peptide mixture was administered to the rabbit (a 100 μg dose) intranasally on day 15.

A 25 μL aliquot of 4 g/L aqueous peptide mixture was administered to the rabbit (a 100 μg dose) intranasally on day 22 along with a 50 μL aliquot of 4 g/L aqueous peptide mixture (a 200 μg dose) intramuscular administration. Blood was drawn from the rabbit on day 22 and ELISA tested for antibody response against the mixture of peptides (1^{st} Bleed).

Blood was drawn from the rabbit on day 36 and ELISA tested for an antibody response against the mixture of peptides (2^{nd} Bleed).

A 25 μL aliquot of 4 g/L aqueous peptide mixture was administered to the rabbit (a 100 μg dose) intranasally on day 50 along with a 50 μL aliquot of 4 g/L aqueous peptide mixture (a 200 μg dose) intramuscular administration.

Blood was drawn from the rabbit on day 57 and ELISA tested for antibody response against the mixture of peptides (3^{rd} Bleed).

A 25 μL aliquot of 4 g/L aqueous peptide mixture was administered to the rabbit (a 100 μg dose) intranasally on day 80 along with a 50 μL aliquot of 4 g/L aqueous peptide mixture (a 200 μg dose) intramuscular administration.

Blood was drawn from the rabbit on day 87 and ELISA tested for antibody response against the mixture of peptides (4^{th} Bleed).

The results of the optical densities of ELISA tests of rabbit blood are provided below in Table 14. Each observation reflects a single triplicate ELISA test of blood for antibody concentration at the defined time.

TABLE 14

| | Pre-Bleed | 1^{st} Bleed | 2^{nd} Bleed | 3^{rd} Bleed | 4^{th} Bleed |
|---|---|---|---|---|---|
| Observation | (day 0) | (day 22) | (day 36) | (day 57) | (day 87) |
| 1 | 0.388 | 0.413 | 1.018 | 0.986 | 1.81 |
| 2 | 0.417 | 0.477 | 0.924 | 0.968 | 1.692 |
| 3 | 0.450 | 0.563 | | | 1.284 |
| 4 | 0.480 | 0.460 | | | |

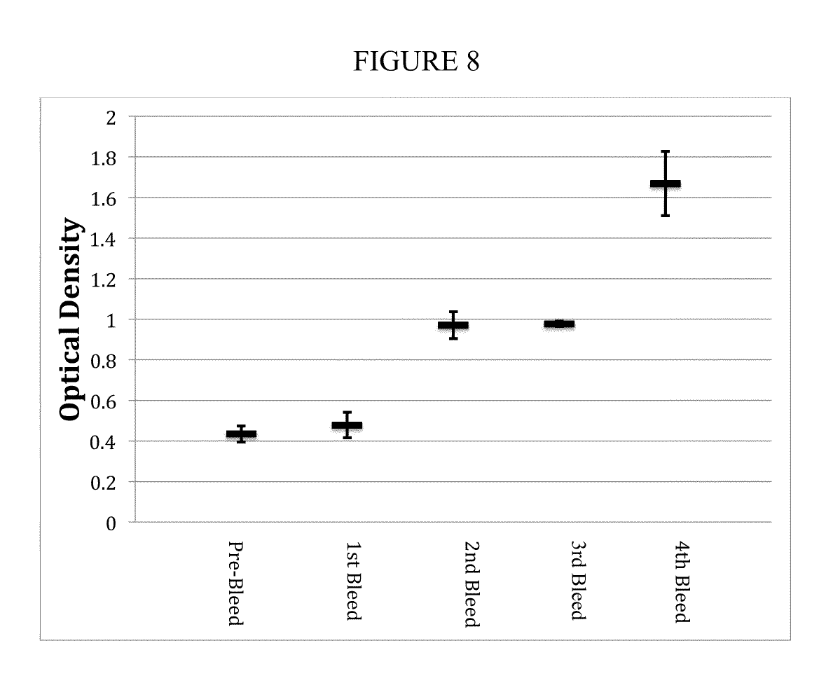

Values for the Pre-bleed and 4^{th} bleed were found to be statistically different at P<0.0074. Values for the 1^{st} and 2^{nd} bleed were found not statistically different. Values for the 2^{nd} and 3^{rd} bleed were found not statistically different. Values for the 3^{rd} and 4^{th} bleed were found to be statistically different at P<0.0411. The means of the data for each column of Table 14 along with standard deviation are presented in FIG. 8. FIG. 8 illustrates the strong immune response of the tested vaccine in rabbit.

The ELISA tests of various bleeds of the rabbit inoculated with the vaccine demonstrate that the rabbit responded with a strong antibody response against the mixture of the peptides of the vaccine. The peptides of the vaccine are conserved Replikin peptides of Ebolavirus or adjuvant homologues thereof. FIGS. 1-5 and 7 and Examples 1, 2, and 6, demonstrate that Replikin sequences of Filoviruses, including Ebolavirus and Marburg virus, are structurally and functionally related to rapid replication, virulence, and lethality in Filoviruses and are conserved across strains of Filovirus, including most virulent strains. Because Replikin sequences are structurally and functionally related to rapid replication, virulence, and lethality in Filovirus, targeting Replikin structures provides a mechanism for reducing rapid replication, virulence, and lethality. The test results provided in Table 14 and FIG. 8, therefore, demonstrate a strong immune response in vaccinated rabbit against Ebolavirus and Marburg virus Replikin peptides and homologues thereof that target rapid replication, virulence, and lethality mechanism of Ebolavirus and Marburg virus. The vaccine is therefore useful for preventing and treating Ebola and Marburg infection by targeting these replication mechanisms.

The vaccine of the example (as well as individual peptides of the vaccine or various mixtures of the individual peptides of the vaccine) is likewise useful as an immunogenic composition for stimulating the immune system of a subject against an Ebolavirus or Marburg virus, against an infection of an Ebolavirus or Marburg virus, or to prevent an Ebolavirus or Marburg virus infection. Antibodies produced from the immune response are likewise useful for diagnosing Ebolavirus and Marburg virus contamination and infection.

The vaccine of the example (as well as individual peptides of the vaccine or various mixtures of the individual peptides of the vaccine) is also useful as a blocking composition to block replication of virus within infected cells.

### Example 4

### Synthetic Replikin Vaccine Against Ebolavirus Using Selected Conserved Peptides

A synthetic Replikin vaccine containing an approximately equal-parts-by-weight mixture of Ebolavirus Replikin peptides SEQ ID NO(s): 1-20, SEQ ID NO(s); 2 and 21-24, SEQ ID NO(s): 2, 4, 5, 9, 13-16, 18-20, 22, and 25-33, SEQ ID NO(s): 1, 2, 4, 6, 7, 10, 16, 19, 26, 27, and 33, or SEQ ID NO(s): 19, 34, and 35 was designed for use against relatively-lethal isolates of Ebolavirus or Marburg virus. The sequences were found to be shared Replikin structures in Ebolaviruses and Marburg viruses across strains and across geographical regions.

The vaccine was engineered to inhibit the lethality of relatively-lethal strains of Ebolavirus and Marburg virus.

The vaccine may be administered to an animal or human susceptible, exposed to, or suffering from infection of a Filovirus, including an Ebolavirus or a Marburg virus. The blocking mechanism of the vaccine provides a therapeutic and prophylactic effect. The immune response generated by the vaccine provides a prophylactic effect. Because each of the sequences comprised in the vaccine is related to rapid replication and lethality of the virus and provides an immune and a blocking response upon administration to a subject at sufficient volume and concentration, each sequence may be used as an individual active component in a vaccine against Filovirus, including an Ebolavirus or a Marburg virus; a mixture of peptides is not necessary to provide an effective vaccine. A mixture of peptide sequences may also be used.

### Example 5

### Synthetic Replikin Vaccine Against Filovirus

A synthetic Replikin vaccine containing an approximately equal-parts-by-weight mixture of forty-one Ebola virus Replikin peptides, Marburg virus Replikin peptides, and adjuvant homologue peptides was designed for use against relatively-lethal isolates of Filovirus, including Ebolavirus and Marburg virus. The vaccine was engineered from sequences confirmed to be conserved across regions (countries) and across time. Conservation was particularly noted in the key amino acid residues of the Replikin sequence, namely, lysine and histidine amino acid residues. The vaccine was engineered to inhibit the lethality of Ebolavirus and Marburg virus.

The vaccine comprises a mixture of the following forty-one Replikin peptides in sterile water: SEQ ID NO(s): 1-41.

The vaccine may be administered to an animal or human susceptible, exposed to, or suffering from infection of a Filovirus. The blocking mechanism of the vaccine provides a therapeutic and prophylactic effect. The immune response generated by the vaccine provides a prophylactic effect.

### Example 6

### Conservation of SEQ ID NO: 19

SEQ ID NO: 19 was identified as conserved in the Filovirus Marburg from 1967 through 2015. The following accession numbers were found to contain sequences comprising SEQ ID NO: 19 in the given year.

TABLE 15

| Year | Accession Number |
|---|---|
| 1967 | ADM72999 position 215, ADM72992 position 215, |
| | ADM72985 position 215, ABS17559 position 215 |
| 1987 | YP_009055226 position 215, ACD13013 position 215, |
| | ACD13006 position 215, ABS17552 position 215 |
| 1992 | CAA78118 position 214, 2110212E position 215 |
| 1994 | P35258 position 215, CAA82540 position 215, CAA45746 |
| | position 215 |
| 1995 | P41326 position 215 |
| 1999 | AFV31322 position 215, AFV31196 position 215, AFV31175 |
| | position 215, AFV31147 position 215, AFV31141 position 215 |
| 2000 | AFV31315 position 215, AFV31308 position 215, AFV31301 |
| | position 215, AFV31294 position 215, AFV31287 position 215, |
| | AFV31280 position 215, AFV31273 position 215, AFV31266 |
| | position 215, AFV31259 position 215, AFV31252 position 215, |
| | AFV31245 position 215, AFV31238 position 215, AFV31231 |
| | position 215, AFV31224 position 215, AFV31217 position 215, |
| | AFV31210 position 215, AFV31203 position 215, AFV31189 |
| | position 215, AFV31182 position 215, AFV31168 position 215, |
| | AFV31161 position 215, AFV31154 position 215 |
| 2003 | AAQ55259 position 215 |
| 2004 | AAR85464 position 215, AAR85457 position 215 |
| 2005 | AKI84284 position 215, AKB09573 position 215, ABA87128 |
| | position 215 |
| 2007 | YP_001531157 position 215, ACT79244 position 215, |
| | ACT79237 position 215, ACT79230 position 215, ACT79216 |
| | position 215, ACT79209 position 215, ACT79202 position 215 |
| 2008 | AFV31336 position 215, AFV31329 position 215, AEW11938 |
| | position 215, ACT79223 position 215 |
| 2009 | AFV31371 position 215, AFV31364 position 215, AFV31357 |
| | position 215, AFV31350 position 215, AFV31343 position 215 |
| 2012 | AGL73419 position 215, AGL73412 position 215 |
| 2014 | AIL25249 position 215 |
| 2015 | AJD39286 position 215, AJD39279 position 215, AJD39272 |
| | position 215 |

### Example 7

### Synthetic Replikin Vaccine Against Marburg Ebolavirus

A synthetic Replikin vaccine containing an approximately equal-parts-by-weight mixture of three Marburg Ebolavirus Replikin peptides was designed for use against relatively-lethal isolates of Marburg Ebolavirus. The vaccine was engineered from sequences confirmed to be conserved across regions (countries) and across time. Conservation was particularly noted in the key amino acid residues of the Replikin sequence, namely, lysine and histidine amino acid residues. The vaccine was engineered to inhibit the lethality of Marburg Ebolavirus

The vaccine comprises a mixture of the following three Replikin peptides in sterile water: SEQ ID NO(s): 19, 34, and 35.

The vaccine may be administered to an animal or human susceptible, exposed to, or suffering from infection of Ebola virus. The blocking mechanism of the vaccine provides a therapeutic and prophylactic effect. The immune response generated by the vaccine provides a prophylactic effect.

### Example 8

### Computer Methods for Determining Virulence and Lethality of Ebola Virus

A prediction of expansion or retraction of virulence or lethality of Filovirus population (including Ebolavirus and Marburg virus) may be performed by a processor. A prediction may be output to a user or display. Likewise, a particular Replikin peptide or Replikin Peak Gene within an isolate or population of isolates of Filovirus may be predicted to be expanding or retracting in replication or lethality and this prediction may be output to a user or display. A machine-readable storage medium may contain executable instructions that, when executed by a processor, cause the processor to provide sufficient data to a user, a printout, or a display such that the user or a user of the printout or display may predict expansion or retraction of population of a Filovirus. A process for prediction may comprise: comparing a Replikin concentration of at least one first isolate of Filovirus with a Replikin concentration of at least one second isolate of Filovirus; and predicting the population of the first isolate to be expanding if the Replikin concentration of the first isolate is greater than the Replikin concentration of the second isolate. In another embodiment, predicting the population of the first isolate to be expanding if the Replikin concentration of the first isolate is greater than four Replikin sequences per 100 amino acid residues.

A computer system may include a processor coupled to a network, and a memory coupled to a processor, wherein the memory contains a plurality of instruction to perform the methods of prediction discussed herein.

A user of outputted data from a processor, storage medium, machine-readable medium, or computer system may include any person or any machine that records or analyzes the outputted data. A display or printout may include any mechanism by which data is outputted so that any person or any machine may record or analyze the outputted data, including a printed document, a visual impulse, an aural impulse, or any other perceivable impulse, a computer monitor, a set of numbers, or any other display or printout of data including a digital recording medium. A computer program product may have computer program logic arranged to put into effect a method of predicting an outbreak of Ebolavirus. The program may be contained in a signal or non-transitory signal.

What is claimed is:

1. A pharmaceutical composition administrable to a subject susceptible to or suffering from infection of a Filovirus, wherein said composition comprises at least one peptide consisting of no more than 50 amino acid residues comprising a sequence of any one of SEQ ID NO(s): 1-13 and 15-41 and a pharmaceutically-acceptable carrier or adjuvant.

2. The pharmaceutical composition of claim 1, wherein said composition comprises at least one peptide consisting of no more than 50 amino acid residues comprising a sequence of any one of SEQ ID NO(s): 2, 4, 7, 9, 15, 18, 19, 20, 26, 28, 31, or 35-41.

3. The pharmaceutical composition of claim 1 comprising at least one peptide consisting essentially of at least one of SEQ ID NO(s): 1-13 and 15-41.

4. The pharmaceutical composition of claim 1 comprising at least one peptide consisting essentially of at least one of SEQ ID NO(s): 2, 4, 7, 9, 15, 18, 19, 20, 26, 28, 31, and 35-41.

5. The pharmaceutical composition of claim 1 comprising at least one peptide consisting of at least one of SEQ ID NO(s): 2, 4, 7, 9, 15, 18, 19, 20, 26, 28, 31, and 35-41.

6. The pharmaceutical composition of claim 1 comprising a mixture of at least two peptides, each consisting of no more than 50 amino acid residues, wherein each of said two peptides comprises at least one amino acid sequence of SEQ ID NO(s): 1-41 that is different from the other of said two peptides.

7. The pharmaceutical composition of claim 1 comprising a mixture of at least two peptides, each consisting of no more than 50 amino acid residues, wherein each of said two peptides comprises at least one amino acid sequence of SEQ ID NO(s): 2, 4, 7, 9, 15, 18, 19, 20, 26, 28, 31, or 35-41 that is different from the other of said two peptides.

8. The pharmaceutical composition of claim 1 comprising a mixture of at least two peptides, each consisting of no more than 50 amino acid residues, wherein each of said two peptides comprises at least one amino acid sequence of SEQ ID NO(s): 2, 4, 7, 9, 15, 18, 19, 20, 26, 28, 31, or 35-40 that is different from the other of said two peptides.

9. The pharmaceutical composition of claim 7, wherein each of said two peptides consists of at least one different amino acid sequence of SEQ ID NO(s): 2, 4, 7, 9, 15, 18, 19, 20, 26, 28, 31, 35, or 39.

10. The pharmaceutical composition of claim 1 comprising a mixture of at least thirteen peptides, each consisting of no more than 50 amino acid residues, wherein each of said at least thirteen peptides comprises at least one amino acid sequence of SEQ ID NO(s): 2, 4, 7, 9, 15, 18, 19, 20, 26, 28, 31, 35, and 39 that is different from all other of said at least thirteen peptides.

11. The pharmaceutical composition of claim 10 wherein each of said at least thirteen peptides consists of a different sequence of SEQ ID NO(s): 2, 4, 7, 9, 15, 18, 19, 20, 26, 28, 31, 35, and 39.

12. The pharmaceutical composition of claim 11, further comprising four additional peptides, wherein each of said four additional peptides consists of a different sequence of SEQ ID NO(s): 36, 37, 38, and 40.

13. The pharmaceutical composition of claim 1, wherein administration of said pharmaceutical composition to a subject with an immune system stimulates an immune response against any one of the amino acid sequences of SEQ ID NO(s): 1-41.

14. A vaccine against a Filovirus comprising the pharmaceutical composition of claim 1.

15. The vaccine of claim 14 against Ebola Reston, Ebola Sudan, Ebola Guinea, Ebola Sierra (Zaire), or Marburg virus.

16. A method of preventing or treating Ebolavirus or Marburg virus infection comprising administering the pharmaceutical composition of claim 1 to a subject.

17. A method of stimulating the immune system of a subject against Ebolavirus or Marburg virus comprising administering the pharmaceutical composition of claim 1 to a subject.

18. An isolated, chemically-synthesized, or recombinantly-generated binding molecule that specifically binds at least one sequence of SEQ ID NO(s): 1-13 and 15-41.

19. The isolated, chemically-synthesized, or recombinantly-generated binding molecule of claim 18 that specifically binds at least one sequence of SEQ ID NO(s): 2, 4, 7, 9, 15, 18, 19, 20, 26, 28, 31, or 35-41.

20. A method of providing passive immunity in a patient suffering from an Ebolavirus infection or Marburg virus infection comprising administering to the patient at least one isolated, chemically-synthesized, or recombinantly-generated binding molecule of claim 18.
